(19) United States
(12) Patent Application Publication
(10) Pub. No.: US 2004/0033554 A1
(43) Pub. Date: Feb. 19, 2004

(54) Automated semi-solid matrix assay and liquid handler apparatus for the same

(75) Inventor: Elaine Powers, High Bridge, NJ (US)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharmaceuticals, Inc, Bridgewater, NJ

(21) Appl. No.: 10/638,976

(22) Filed: Aug. 12, 2003

Related U.S. Application Data
(63) Continuation of application No. PCT/US02/03297, filed on Feb. 5, 2002.
(60) Provisional application No. 60/269,645, filed on Feb. 16, 2001.

(30) Foreign Application Priority Data
Jul. 20, 2001 (GB) 0117706.2

(51) Int. Cl. C12Q 1/02; B01L 3/00

(52) U.S. Cl. 435/29; 422/100

(57) ABSTRACT

An improved liquid handling machine capable of regulating the temperature of assay compounds in the automated preparation of culture trays for biological assays is disclosed. The machine includes a horizontally movable table positioned beneath a vertically movable head. The table is divided into a plurality of stations holding mixing trays, culture trays and reservoirs of liquid assay compound. The head holds a plurality of pipettes which aspirate and expel liquid to transfer and mix the assay compounds between the reservoir, the mixing trays and the culture trays upon coordinated movement of the head and the table as controlled by a microprocessor. Each station on the table has independent heating, cooling and temperature sensing elements for regulating the temperature of the liquid held in a tray or reservoir at the station. A device for automatically evaluating the results of the assay, such as by fluorescence, spectrophotometric or radioactive techniques is incorporated with the improved liquid handling machine.

### FIELD OF THE INVENTION

The present invention relates to methods for automated preparation of multi-well plates containing semi-solid matrices, such as those used for soft agar assays, and methods for automating semi-solid matrix assays. The present invention also relates a liquid handler suitable for preparing the same.

### BACKGROUND OF THE INVENTION

Rapid, high throughput assay techniques result in a large number of potential hit compounds, but secondary assay methods used to confirm the biological significance of such hits have not been automated, resulting in a bottleneck at the secondary assay level and significantly slowing down compound development.

Agar is a generic name for a class of compounds generally defined as a dried mucilaginous substance extracted from red algae, having the property of melting at about 100 deg. C. and solidifying into a gel at about 40 deg. C. Agar is not digested by most bacteria and is used as a gel in the preparation of solid culture media. Dorland's Illustrated Medical Dictionary, 25th ed., W. B. Saunders Co. 1974. Agarose is a modified agar, whereby sugars, methyl groups, and other chemical groups are chemically bonded to agar in order to enhance desired physical properties, such as low gelling temperature.

A soft agar assay typically involves more than one layer of semi-solid matrix. It is generally preferred that two or more layers are used, where each is preferably composed of either agar or agarose mixed with a liquid nutrient medium. It is also preferred that each layer is usually of about equal volume. The bottom layer typically has a slightly higher concentration of semi-solid matrix, such as agarose, and contains everything contained in the top layer except the cells. The test compound may be in both, or either, layers or alternatively, added after the semi-solid matrix has solidified.

Soft agar assays were originally developed as secondary assays for culturing cells requiring three-dimensional non-attached, or non-adherent, cell growth and were originally developed for culturing bone marrow and leukocytic cells in vitro. A standard in vitro method used to quantitate the number of macrophage precursors in bone marrow is to grow bone marrow stem cells in soft agar with growth media and supplemented with various growth factors, such as colony stimulating factor-1 (CSF-1). After an incubation period, the colony-forming units of cells (CFUs) are counted.

Eventually, techniques for culturing non-adherent cells in semi-solid matrices, such as soft agar assay, were applied to tumor cells as in vitro assays predictive of a tumor cells' responsiveness to chemotherapeutics in vivo. In such studies, tumors are removed from a patient, and cut into smaller fragments. After further dissociation of tumor fragments into individual cells, the individual cells are plated within a semi-solid matrix. However, only a subpopulation of tumor cells, called stem cells, was found capable of in vitro proliferation and colony formation, or clonal growth, in soft agar. Consequently, this assay was called a tumor stem cell assay, or alternatively, tumor clonogenic assay.

As currently used, there is considerable variation in the methodology used in such soft agar assays. For example, the most frequently used semi-solid matrices are agar and agarose, and are commonly referred to as just “agar”. The term ‘colony’ is determined by the researcher, which may be defined by the number of cells or total diameter. Typically, a colony is defined as consisting of at least 40 or 50 cells, although sometimes as few as 30 cells or less, with smaller aggregates frequently referred to as ‘clusters’. The incubation period required for a given cell type to reach the critical size or number of cells to be called a ‘colony’ varies between cell types, but typically requires an incubation period of between seven-to-fourteen days. Therefore, the incubation period in a given study varies, with longer periods being used if the cell growth is slow, or varying the cells defining a ‘colony’. If diameter is used as the defining criterion, a ‘colony’ is typically defined as being 50 microns. Another point of variability in soft agar assays is deciding what is an ‘effective’ dose of a test compound, for example 50% inhibition, 70% inhibition, or any inhibition.

Several culture techniques have been developed for growing non-adherent cells in semi-solid matrices. Cells cultured in vitro require a variety of growth factors to either promote growth or maintain viability. Numerous types of growth media are commercially available, such as Dulbecco's Modified Eagle Medium, RPM1, Ham's F12 and contain a wide variety of growth-requiring factors. In addition to growth media, many factors are provided as supplemental growth factors, such as fetal calf serum or fibroblast growth factor. Growth media and supplemental factors may be added directly to the semi-solid matrix while the matrix is still liquid, or after the semi-solid matrix has solidified, and are thought to promote cell growth and/or division. Unfortunately, it is not known how these various factors affect cell sensitivity to therapeutic agents (Singletary, S. E., et al., 1985). The human tumor stem cell assay revisited. International Journal of Cell Cloning 3:116-128). Availability of commercial ‘2× medium’ reduces/replaces need for many supplements—original assay designs included conditioned medium, high levels of serum (15-20%), or supplemental factors beyond the usual growth medium components, which is important since diluting the growth medium by more than 10% has been shown to reduce cell growth. (Singletary 1985, ibid).

Soft agar assays are frequently used as secondary assays as a cell model system for assessing the effects of test compounds on tumor growth. A number of different protocols for soft agars have been established, and include the Hamburger-Salmon, Courtenay-Mills, Courtney-Mills plus additions, soft agar (no additions) and soft agar plus additions, see, for example, West, C. M. L, and Sutherland, R. M., Int. J. Cancer 37:897-903 (1986). Tumor cells suspended in agarose or other types of agar are capable of unattached growth, resulting in formation of three-dimensional colonies. Such colonies are thought to more closely mimic natural tumor formation in vivo.

The two most commonly used soft agar assay techniques are referred to as the Hamburger-Salmon (or H-S) and the Courtenay-Mills (or C-M) methods. In the Hamburger-Salmon assay, enriched media are added to agar with the nutrients being added to both layers, with cells included in the top layer. The bottom layer consists of about 0.5% agar and the top layer is of about 0.3% agar. The cultures are plated in dishes (plates containing one to 24 wells per plate).

An alternative method is described by Courtenay-Mills, see for example, Courtenay, V. D. 1984, A replenishable soft agar colony assay for human tumour sensitivity testing. Recent Results in Cancer Research 94:17-34, in which red blood cells are added to the agar as a component in the agar layers, requires the addition of liquid medium about every 5 days, a low 02 atmosphere, and uses culture tubes, instead of plates. Tveit, K. M., et al., 1989 Colony-forming ability of human ovarian carcinomas in the Courtenay soft agar assay. Anticancer Research 9:1577-1582, report that human ovarian carcinoma cells formed more tumor-derived colonies in the C-M assay than the H-S method. However, part of the improvement may be explained by the opportunity for longer culture times with the C-M method due to replenishing the growth media during the culture period.

A number of studies have been done to compare the sensitivity of the two culture methods. For example, comparison of tumor cell lines and cells isolated from tumors demonstrated that cells had greater sensitivity to chemotherapeutics in the H-S method than the C-M method, even though the C-M method had a greater plating efficiency (see Endresen, L., et al. 1985. Chemosensitivity). Measurements of human tumour cells by soft agar assays are influenced by the culture conditions. Br. J. Cancer 51:843-852; Tveit, K. M., et al., 1981. Comparison of two soft-agar methods for assaying chemosensitivity of human tumours in vitro: malignant melanomas. Br. J. Cancer 44:539-544; West, C. M. L. and Sutherland, R. M. 1986. A radiobiological comparison of human tumor soft-agar clonogenic assays. International Journal of Cancer 37:897-903).

Methylcellulose may be added to the assay to facilitate harvesting of the colonies, but only if the cell growth is not affected by methylcellulose's inhibiting growth effects (Stanisic, T. H., et al., 1980. Soft agar-methylcellulose assay for human bladder carcinoma. Cloning of Human Tumor Stem Cells, pp.75-83). Addition of methylcellulose is traditionally used when culturing erythroid progenitor cells. The viscosity of methylcellulose prevents aggregation of the cells, but is not enough to hold the cells in place (Metcalf, D. 1984. The hemapoietic colony stimulating factors. Elsevier Science Publishers, New York); in contrast, agar and agarose, even at as low a percentage as 0.3%, form true gels.

Various semi-solid substrates have been examined. In one study of lymphoma cells, purified agarose was found to be superior for the assay than agar or methylcellulose for determining colony formation and inhibitory effects (Hays, E. F., et al. 1985. Conditions affecting clonal growth of lymphoma cells in a semisolid matrix. In Vitro Cellular & Developmental Biology 21 (5):266-270). Agarose, which is a modified agar with most of the large charged molecules removed, may allow for better diffusion of growth factors than agar with its charged matrix molecules.

To circumvent the problem of needing larger well volume for the soft agar assays, researchers have tested other semi-solid substrates to mimic cell growth in soft agar. As an example, in a study reported by Fukazawa, H., et al. 1995, a microplate assay for quantitation of anchorage-independent growth of transformed cells. Analytical Biochemistry 228:83-90, poly(HEMA)-coated 96-well plates were assessed by tetrazolium dye reduction or 3H-thymidine incorporation. Poly(HEMA) is an anti-adhesive polymer with results similar to soft agar (only looking at cell growth, no compound effects).

Another adaptation to the soft agar assay is to quantitative colonies by use of 3H-thymidine incorporation into DNA as the cell divides, instead of relying on visual counting to determine the number of colonies, see Unshared, G., et al., 1985. A new technique to register proliferation of clonogenic cells from brain tumors. Journal of Near-Oncology 3:203-209. In this particular assay, red alga extract Furcellaran was used as the substrate gel in a single layer as opposed to the double layer semi-solid matrix system. One disadvantage of radiolabeling as a measurement method is that requires additional steps of harvesting each tube's contents onto glass filters and counting the material in a liquid scintillation counter, as well as requiring the use of radioactivity.

Dissociated tumor cells may be exposed to a variety of experimental treatments, such as chemotherapy or radiation, either prior to plating or by being plated with the chemotherapeutic compound in the semi-solid matrix. The soft agar assay may be more predictive for a cell's resistance than for its sensitivity; for example, the accuracy of predictions were 96% and 91% for resistance, and 62% and 59% for susceptibility as reported in Salmon, S. E., et al., 1980. Clinical correlations of drug sensitivity in the human tumor stem cell assay. Recent Results in Cancer Research 74:300-305 and Scholz, C. C., et al., 1990. Correlation of drug response in patients and in the clonogenic assay with solid human tumour xenografts. Eur. J. Cancer 26 (8):901-905, respectively. Other researchers and we have observed that cells grown in three-dimensional structures are more resistant to chemotherapeutics than the same tumor cells grown as a monolayer (O'Connor, K. C. 1999. Three-dimensional cultures of prostatic cells: tissue models for the development of novel anti-cancer therapies. Pharmaceutical Research 16(4):486-493). Occasionally however, cells grown in a 3-dimensional structure may be more sensitive to a compound, as shown by Hedlund, 1999. Three-dimensional spheroid cultures of human prostate cancer cell lines. The Prostate 41:154-165, with PC-3 cells in spheroids treated with 1,25(OH)₂ D₃. Results such as these demonstrate that cells grown as monolayers are very different than those grown in 3-dimensional structures and, consequently, that cells in a monolayer are very different from cells within a tumor.

In addition to examination of anti-cancer compound effects, the culturing of cells on soft agar in vitro has been used to examine the effects of growth factors on tumor cells (for example Rizzino, A. 1987. Soft agar growth assays for transforming growth factors and mitogenic peptides. Methods in Enzymology 146:341-352).

Cells capable of proliferating in semi-solid matrices such as soft agar may not reflect all tumor cell subpopulations. Tveit, K. M., et al. 1985. Selection of tumour cell subpopulations occurs during the cultivation of human tumours in soft agar. A DNA flow cytometric study. Br. J. Cancer 52:701-705 demonstrated that using the C-M method, cultivation in soft agar selected specific aneuploid tumor cell populations. Despite these drawbacks, results obtained from soft agar assays are thought to be predictive of a particular compound's inhibitory effects in vivo, since a compound must be able to kill the tumor stem cells to be effective in stopping tumor growth.

As currently being practiced, soft agar techniques are labor intensive. Determination of colonies grown in the soft agar assay has traditionally been scored visually, where the number of colonies is counted by eye, or the colonies are stained and counted with the aid of an imaging system. Therefore, this quantification may be distorted by subjective counting, such as differentiating between clusters and colonies, and inaccuracies in the counting by imagers produced by the 3-dimensional nature of the agar culture (for review, see Singletary, S. E., et al., 1985. The human tumor stem cell assay revisited. International Journal of Cell Cloning 3:116-128). See, for example, Salmon, S. E, et al, Int. J. Cell Cloning 2:142-160 (1984). The visual counting is very labor-intensive and subjective. Salmon et al report that most technicians have substantial difficulty doing colony counts for more than three to four hours per day, principally because of operator fatigue. The staining method is limited by the imager's ability to identify discrete colonies; overlapping colonies interfere with an accurate count. Colonies that touch or overlap visually, even though they are different depths, may be scored inappropriately by the imager. Despite these problems, imagers are routinely used to increase the counting speed and reduce labor for the soft agar assay. One such imaging system, as described in Salmon, S. E., et al., 1984. Evaluation of an automated image analysis system, an Omnicon FAS II image analysis system, for counting human tumor colonies. International Journal of Cell Cloning 2:142-160, was able to count the plates on trays ten times faster than experienced technicians were with good correlation with technician counts. These methods of quantification need large surface areas for visualization, requiring the use of 24-well multiwell places or larger. This prevents the use of the automated 96-well systems.

Traditional assays determine the presence or absence of colonies, and typically do not account for the cell viability within a colony. The method presented herein may optionally be used with viability staining for determination of the cell status.

Other concerns have been raised about how representative the soft agar assay is to the tumor growth in vivo. The effect of a test compound on a proliferating population of tumor cells may not be representative of its effect on the total tumor cell population in vivo. Studies on tumor cell viability and plate density on the growth parameters, see for example, Page, R. H., et al. 1988. When the cell population is derived from a tumor, sufficient numbers of cells must be isolated for successful culturing. Unfortunately, the dissociation techniques used to produce the single cell population may damage the cells resulting in fewer viable cells, and distort the efficacy of a particular test compound. In addition, not all the cells isolated from the tumors are capable of non-adherent growth in semi-solid matrices. Since limited cell populations obtained from a given tumor, such as tumor stem cells, proliferate in semi-solid matrices, and a given tumor may contain differing levels of stem cells when excised, results obtained from a soft agar assay may vary.

In a soft agar assay, cells are held in situ within the semi-solid matrix, and a colony forms by cell division. An alternative assay, the spheroid assay, colonies form by aggregation of individual cells. Both are deemed to be more representative of the in vivo tumor than monolayer cultures, because tumors grow in a three-dimensional structure with uneven distribution of nutrients and oxygen in the immediate area.

Spheroids were initially developed with aggregates of embryonic cells, then subsequently used for tumor cell culture (for review, Santini, M. T. and Rainaldi, G. 1999. Three-dimensional spheroid model in tumor biology. Pathobiology 67:148-157). Spheroid cultures are done by several methods. The most commonly used is the liquid-overlay method, which involves placing a single cell suspension in dishes coated with a non-adhesive surface, such as agar or agarose. Aggregates usually begin forming within one-to-three days. The spheroids must be separated from any remaining single cells and transferred into a second dish. To produce spheroids, single cells may also be seeded into a Spinner flask and kept in suspension by stirring. Similarly, the cells may be rotated in non-adhesive flasks on a gyrator or rotating-wall vessels (O'Connor, K. C. 1999. Three-dimensional cultures of prostatic cells: tissue models for the development of novel anti-cancer therapies. Pharmaceutical Research 16(4):486-493).

The soft agar assay has several advantages over spheroid cultures assays. For example, the cells are not subjected to the liquid sheer forces present in the stirring or rotating flasks. Because spheroids must be separated from the cells not forming spheroids, spheroid assays increase the handling of the colonies as well as being laborious. The soft agar assay permits both concentration increase, and depletion, of growth factors, etc. in the immediate microenvironment surrounding the colony, which may be more representative of the in vivo tumor situation. Also, the factors present in the microenvironment around a soft agar colony may affect the test compound, which would affect the compound's abilities at inhibiting cell growth. The soft agar assay also allows a given colony to produce extracellular matrix (ECM) components, in turn, permitting intracellular signaling which may be similar to those produced by a tumor in vivo. ECMs have been observed in spheroids as well, but the ECMs expressed and/or secreted in either assay may differ from those produced by a tumor in vivo. The effect of contact with the semi-solid matrix, such as agar or agarose, on colony formation is unknown and this variable is not present in a spheroid assay. The length of time for the formation of colonies is comparable between the two techniques.

A semi-solid matrix holds the cells in situ, thus permitting continuous observation of a single cell or individual colony. Because of the continuous mixing typical for a spheroid assay, the spheroids are not held in place, making it difficult to keep track of a single colony. Even when the spheroid assay does not require movement, the liquid nature of the assay permits spheroid movement over time.

In situations where it is advantageous to have aggregates consisting of more than one type of cell, a spheroid assay is preferred over a soft agar assay, where the colonies arise from single cells. For example, O'Connor reports on studies examining the effects of metastatic prostate cells, PC-3 cells, on osteoblast-like cells, see O'Connor, K. C. (1999) Three-dimensional cultures of prostatic cells: tissue models for the development of novel anti-cancer therapies. Pharmaceutical Research 16(4):486-493.

Despite the drawbacks of semi-solid matrix assays, such as those described above and being labor intensive, the soft agar assay is still routinely used as a secondary assay, and is preferred over monolayer culturing of cells because it is thought to more closely mimic biological tumor formation.

Potential Uses for Automated Semi-Solid Matrix Assays

As described herein, the present assay is useful for any semi-solid matrix assay for examining non-adherent cell growth with any optically active reporting system as well as radioisotope systems. The present assay is described herein as being adapted for a 96-well plate, but the present assay may also be adapted for use with greater number multi-well formats, such as 384-well or 1536-well plate formats.

Semi-Solid Matrices

Semi-solid matrices include gels suitable for culturing cells. Preferred semi-solid matrices are those capable of sustaining growth of cells, and preferred forms of semi-solid matrices are forms of agar, including modified forms such as agarose.

Typically, the preferred semi-solid matrix for use in the present invention forms a liquid at temperatures above room temperature or above the temperature required to incubate the cells, and forms a semi-solid, or gel, when at about room temperature or the temperature at which the cells are incubated. Preferred forms of semi-solid matrices are agar and agarose. However, a wide variety of polymers, including proteins and their derivatives, may be used as semi-solid matrices in the present invention. Matrigel®, collagen or gelatin, or other similar materials may also be used as the semi-solid matrix.

Agar is a generic name for a class of compounds generally defined as a dried mucilaginous substance extracted from red algae, having the property of melting at about 100 deg. C. and solidifying into a gel at about 40 deg. C. Agar is not digested by most bacteria and is used as a gel in the preparation of solid culture media. Dorland's Illustrated Medical Dictionary, 25th ed., W. B. Saunders Co. 1974.

Agarose is the neutral linear polysaccharide fraction found in agar preparations, generally comprised of D-galactose and altered 3,6-anhydrogalactose residues, thus agarose is a modified agar. Agarose is a purified linear glactan hydrocolloid isolated from agar or agar-bearing marine algae. Agarose forms a gel matrix when it is at it's gel point, which may be different than it's melting temperature. Typically, sugars, methyl groups, and other chemical groups are chemically bonded to agar, or fraction derived from agar, in order to enhance desired physical properties, such as low gelling temperature.

Alternative semi-solid matrices include polymers which form a relatively firm matrix, such as poly(HEMA) which is an anti-adhesive polymer.

Collagen is a major mammalian protein of the white fibers of connective tissues, cartilege, and bone. Collagen is generally insoluble in water, but is typically altered to improve desireable properties such gelling at a given temperature.

Gelatin is a derived protein formed from collagen of tissues by boiling in water. Gelatin swells up when put in cold water, but dissolves in hot water.

In the case of Matrigel®, collagen or gelatin, or other similar materials, temperature control of the material is important to prevent premature gelling when the material warms to about room temperature. Therefore, with such materials, it is important to keep the materials chilled prior to filling the wells.

The wells or microtubules in the plate may be contain a single layer of the semi-solid matrix, or contain multiple layers. In a preferred embodiment, each well contains multiple layers of the semi-solid matrix are used. In another embodiment, the wells or microtubules may contain layers of different types of semi-solid matrix, for example, the bottom layer may comprise gelatin, and an upper layer may comprise a gelatin-agarose mixture. Where different types of semi-solid matrix are used in the wells or microtubules, it is preferred that the bottom layer is a denser semi-solid matrix than the density of the upper layers.

The same, or different, type of semi-solid matrix may be used to form any or all of the layers. Different types of semi-solid matrices include mixtures comprising the first type of semi-solid matrix, or may be a different substance than that used in the first semi-solid matrix layer, for example, if the first semi-solid matrix is agar, the second semi-solid matrix layer may be a mixture of agar-agarose, or agarose, polymer, collagen or gelatin.

In assays with multiple layers of semi-solid matrices, it is preferred that the first or bottom layer comprises a denser, or firmer semi-solid matrix. This may achieved by use of a slightly higher proportion of semi-solid matrix than the upper layers, or alternatively, by use of a different type of semi-solid matrix which forms a less dense semi-solid matrix relative to the first semi-solid matrix.

Preferred embodiments of the semi-solid matrix assay involves more than one layer of semi-solid matrix, and that one or more semi-solid matrix is mixed with growth medium. It is also preferred that each layer of semi-solid matrix contain growth medium. It is also preferred that each layer is usually of about equal volume. It is preferred that the bottom layer of semi-solid matrix has a slightly higher concentration of semi-solid matrix, and contains everything contained in the top layer of semi-solid matrix except the cells. The test compound and/or cells may be in any or all layers of the liquid semi-solid matrix, or alternatively, are added after the semi-solid matrix has solidified. It is preferred that the cells are added to, or added as part of the top layer of semi-solid matrix.

It is preferred that cells are suspended throughout the top layer so that when a colony of cells forms from a single cell undergoing mitosis, the resulting colonies are also even dispersed throughout the semi-solid matrix. This facilitates visual counting so that the colonies do not touch or overlap each other after the incubation period. Therefore, it is preferred that colonies are suspended throughout the top layer of the semi-solid matrix.

Types of Suitable Cells

The present assay may also be used for determining the number of macrophage precursors in bone marrow as well as examining tumor cells growth. Clonogenic tumor cells have been and may be examined for prediction of individual clinical responses to chemotherapy, screening of new anti-neoplastic drugs, examining gene therapy, and in basic research.

As described herein, the present assay is useful for the examination of any type of cell capable of non-adherent growth. As such, the assay is particularly useful for culturing tumor cells, but may also be used to examine many normal cells, including but not limited to, mammalian stem cells and bone marrow cells. Use of mammalian cells is preferred, and human or primate cells are particularly preferred. Assay methods for such cells are well known, see for example, Vescovi, A. L., (1999) Exp. Neurology 156:71-83. Stem cells are particularly important as they are capable of differentiating into a number of different cell types. Methods for isolating and culturing stem cells are discussed in Murray, K, and Dubois-Dalcq, M., J. Neurosci. Res. 50:146-156 (1997), Pincus, D. W., et al., Ann Neurol. 43:576-585 (1998), Sabate, O., et al, Nature Genet. 9:256-260 (1995), and Svendsen, et al., Exp. Neurol. 148:135-146 (1997).

Another utility for the present invention is to semi-automate, or automate embryonic stem, or other mammalian, cell experiments for genetic manipulation. For example, the present method may permit rapid plating of embryonic stem cells for gene targeting using bacteriophage lambda vectors, the manipulation of which is described in Tsuzuki, T., and Rancourt, D. E., Nucl. Acids Res. (1998) 26(4):988-993. These methods are particularly desirable as a means of allowing targeted mutagenesis mammalian germline without restriction enzymes. Using the present methods, large numbers of stem cells or other mammalian cells may be plated automatically, thus reducing the amount of time to perform and saving labor expenses. In addition, automation reduces human error, and improves reproducibility.

Culture Conditions

Methods for growing cells on semi-solid matrices are well known by those skilled in the art. Generally, cells are capable of stasis or growth in the presence of a growth medium. Such growth media are readily commercially available and suitable for culturing a wide variety of cell types. Well known growth media include complete media available as powdered media or liquid media, for example, Minimum Essential Medium Eagle Medium, either of which may be supplemented with sera, such as 10% Fetal Bovine Serum. Growth media includes the use of deficient media, where one or more nutrients is deleted. Growth media includes serum-free or serum-reduced media, as well as salt mixtures, such as Hank's Balanced Salts. Growth media also includes specialty media which are designed to promote growth of specific cell types.

Preferred growth media are concentrated forms of growth media, such as 10× Dulbecco's Modified Eagle's Medium or Dulbecco's Phosphate Buffered Saline.

Sera includes the use of complete sera and sera replacements or substitutes, such as bovine embryonic fluid.

Growth media may include additional antibiotics, attachment and matrix factors which are usually added to facilitate attachment and spreading of many types of anchorage dependent cells. Buffers may also be added to growth media in order to maintain pH levels. Growth factors such as fibroblast growth factors (FGFs), granulocyte colony stimulating factor (G-CSF), and the like, may also be added to growth media.

Types of Test Compounds

The present assay may also be used to determine the ability and effect of a wide variety of test compounds on the growth or stasis of cells. Suitable test compounds include nutrients, growth factors and/or any other molecule capable of diffusing through the medium to reach the cells suspended in the semi-solid matrix. Test compounds for use in the present invention include naturally-occurring biological compounds, including: viability, surface receptors and proteins, growth factors, secreted proteins, attachment factors, apoptosis/necrosis factors, calcium and other intracellular ions, differentiation agents, substrate components, and glycoproteins. Non-naturally-occurring chemicals, proteins, small molecules and the like may also be used as test compounds in the present assay. In addition, the effects of environmental conditions such as uv light, heat, and so forth, may be tested.

Test compounds also includes compounds such as antibiotics, attachement and matrix factors, buffers, and growth factors such as FGFs, G-CSF, and the like, hormones, lectins, lipopolysaccharides, lipids, amino acids,

Determination of the Effects on the Growth of the Cells

Detection methods that could be utilized to determine the biological effect of such compounds or conditions on the test cells in the present invention include fluorescence, colorimetric substrates, chromogenic enzyme substrates, isotopes, luciferase, green fluorescent protein (GFP), glycosidase enzymes, viability dyes, reactive oxygen species detectors, Ca2+ and Mg2+ and other ion indicators, inorganic ion indicators, pH indicators, etc.

The effects of test compounds on the growth or statis of cells may be determined by a variety of standard techniques used to determine cell viability. Prefered techniques include readily automated detection methods, such as direct and indirect fluorescence and Enzyme-Linked Fluorescence. Currently available fluorescent labels include R-Phycoerythrin, Texas Red Sulforhodamine, BODIPY series, Oregon Green, Fluorescein (FITC), Rhodamine (TRITC), Tetramethylrhodamine, YOYO-1, DAPI, Indo-1, Cascade Blue, Fura-2, amino methylcoumarin, FM 1-43+Lipid, DilC18, NBD, Carboxy-SNARF-1, Lucifer Yellow, Dansyl+R-NH2, Propidium Iodide, Aequorin, sodium sensitive dyes, and the Alexa Dye series (Molecular Probes). Protocols for use of these labels as detection probes are well known.

The effects of different types of cells on the growth or stasis of other cells may be tested. For example, stimulation of cell differentiation of a stem cell, or progenitor cell, may be tested. The present method permits determination of the effects of secreted substances, where an agar or agarose layer is added to separate the different types of cells. The semi-solid matrix layer would prevent the actual physical contact of the different cells, but permits passage of secreted factors.

The preferred colony detection method is to determine cell viability, and the preferred viability method is a fluorescent viability dye, but other viability methods may be used. The preference is for viability dyes is due to the readily available commercial fluorescent plate readers. The preference for viability determination is due to obtaining more biologically-significant information about the assay results, than if just visually counted. For example, viability determination permits distinctions between live cells, alive but static, or dead. As a result, the preferred method permits more information to be determined about the state of tumor cells, and therefore, permits a better understanding of the biological effects of the test compound in each experiment.

Order of Adding Components

It is also well known that modifications of the parameters described in detail herein, such as the number and depth of the agarose layers, may be increased or decreased according to choice of well size and dimensions of the tray used. As described in the instant assay, soluble agents may be added to cultures by layering on top of the semi-solid matrix, in addition to adding such agents directly in the semi-solid matrix before the matrix solidifies. Extracellular matrix proteins and compounds/factors may be incorporated into, or on top of, a semi-solid matrix layer. Materials other than the typical semi-solid matrices such as agar or agarose may be substituted as layers to examine cell growth in this automated method. Unlike agar, which needs to be kept warm to prevent gelling, substrates such as Matrigel®, collagen and gelatin need to be kept cold to prevent gelling. Techniques for using such materials are well known in the art, see for example, Becton Dickinson protocol for use of Matrigel® and Rockland Inc. protocol for Extended Collagen Information Sheet.

Cell migration may also be examined using the instant method. Attractants may be placed in the over- or underlying agarose layers, thus permitting studies on the ability of cells to migrate though the agarose. The agarose layers may be separated to determine the numbers of cells in each layer, or the depths of the layers may be varied to study different distances on migration or secretion ability.

Cell Density

Clonogenic assay conditions are optimized for cell density for the selected detection method. One reason for preferring even distribution of cells within the semi-solid matrix is that touching colonies, by virtue of ECMs or cell permeability, may alter compound/factor perfusion around the colonies, thus distorting the assay results. Therefore, there is a need to provide a clonogenic method that avoids the problem of overlapping or touching colonies. For visual counting, when colonies form too close to one another in a plate, detection of individual colonies is difficult. Consequently, traditional clonogenic assays using visual counting are done with a maximum of 24 wells per plate; this permits sufficient dispersion of the colonies to enable ready visual detection.

Incubation Period

Visual counting, either done with or without the aid of a microscope, is tedious and costly. An alternative, automated detection method would be preferred for convenience, cost, reliability and speed. Colonies are allowed to grow from a single cell, until each forms a colony of sufficient size to be counted. A colony is traditionally defined as containing a minimum of about 30 to about 50 cells or possessing a diameter of 10 microns. In order to achieve this growth or size, it is usually necessary that the incubation period for cell growth for about seven to about fourteen days. Longer periods are not generally preferred because the original growth medium is depleted by the cells during the incubation period. However, if additional growth media and factors are added during the incubation period, the incubation period may be lengthened accordingly.

Plates or Trays, or Microtubes

It is preferred that semi-solid matrix assay is adapted for use with higher number of wells format plates, e.g., multi-well plates. Standardized plates for cell culture work are commercially available with one, three, six, twelve, twenty-four, forty-eight, ninety-six, 384-, 1536-wells and even larger number of wells per plate. Such plates, alternatively known as trays, are readily adapted to the standard footprint of commercially available liquid handlers and other equipment. As the number of wells per plate increases, the volume of each well decreases. Use of higher number of well format plates, such as 96-or 384-well plates, results in significant labor, standardization and cost savings over a traditional 24-well plate format and are preferred.

Preferred plates for use in the invention are those suitable for cell culture work, with multi-well plates being especially preferred. Preferred multi-well plates include those with 96-, 384-, and 1536-wells per plate. It is also preferred that the bottom of wells on the plates are of clear plastic, which facilitates detection methods.

Cost Savings, Advantages

Use of a smaller well size results in significant cost savings of materials per well, as well as requiring use of less overall test compound per well. These factors are important criteria when routinely testing large numbers of compounds, and are even more important when the preference is to perform each concentration in triplicate. The standard visual soft agar assay is performed in 24-well plate (alternatively called a tray) with at least 400 uL per layer, and is traditionally done in triplicate, with a sufficient range of test compound to permit calculation of an IC₅₀ value. The minimum volume of liquid is determined by the size of the plate or tray being used, and is the volume necessary to form a single layer evenly across the well. Generally, about six to about eight different concentrations of test compound are used in a given assay for each compound. Therefore, about eighteen wells are required to determine the IC₅₀ value for a single test compound. The use of larger wells obviously requires concurrently larger volumes of each material added to each well.

In contrast, a 96-well plate format permits use of about 20% of the test compound compared to the amount of material used for a 24-well plate format. For example, to plate triplicate wells at 50 uM using a 10 uM test compound stock, the 24-well plate would require 12 uL of test compound. In contrast, the 96-well plate would require only 2.25 uL of test compound.

The present semi-solid matrix method also permits use of fewer plates for the same number of data points. For example, an experiment examining three test compounds at eight concentrations in triplicate with three control wells (preferably with control wells in each plate), requires the use of four 24-well plates. In contrast, the same experiment using the instant method would only require a single 96-well plate. The cost savings of just the number of culture plates used is considerable. A single 96-well plate currently is listed in the Fisher catalog as $2.74 per plate, whereas four 24-well plates (each at $2.02) currently costs $8.08. This results in a savings of $5.34 per experiment for three test compounds, or about a third of the cost using traditional 24-well plate format. This not only saves money in laboratory supplies, but also reduces space in refrigerators or incubators.

Labor and Time-Saving Significance

Using the present automated system soft agar method, a single full-time employee can prepare assay reagents, plate the cells, add compounds, stain the cells and quantitate the viable cells by fluorescence on the Cytofluor for 5 cell lines, 15 compounds at 8 concentrations in triplicate in 1.5 days. Using the traditional method, this work would take one person eleven full work days. Therefore, the automated multi-well semi-solid assay method described herein requires about one-tenth the man hours of the traditional visual soft agar assay.

The present invention provides an automated, or semi-automated, soft agar assay that determines and quantifies colony viability. Therefore, this assay permits determination of colony formation, similar to the traditional soft agar method, but in addition, permits determination of the effects of compounds, growth factors and the like, on already formed colonies. The present method, therefore, provides superior assay method for identification of growth-inhibiting agents.

Liquid Handler Properties

The present invention is directed to a method of automating preparation of plates with semi-solid matrices. The present invention may be used with existing liquid handler machines which don't have the means to regulate the temperature of a liquid, but it is preferred that the liquid handler machine has a means to regulate temperature of a liquid.

However, any liquid handling machine would work provided the machine has sufficient speed to deliver the reagents quickly and can be operated sterilely. Important features for selecting an automated robot or machine for the present clonogenic method are as follows. The robot or machine should be able to perform serial dilutions of almost any ratio, perform serial dilutions of eight or twelve wells across, deliver precise amounts of liquid, mix the contents of a microplate, be used with microplates of different heights, fill microplates with diluent, transfer samples between microplates.

If the liquid handler machine doesn't have the means to regulate termperature of a liquid semi-solid matrix, then the liquid handling machine must be capable of delivering the matrix to the plate with sufficient speed to prevent the matrix from prematurely gelling. If the plates prepared by the instant method are intended to be used for culturing cells, it is also important that the liquid handler machine is capable of operating sterilely in order to prevent contamination of the plates. The preferred liquid handler should be capable of being programmed to perform serial dilutions of almost any ratio, to perform serial dilutions of eight or twelve wells across, to deliver precise amounts of liquid, to mix the contents of a microplate, to be used with microplates of different heights, to fill microplates with diluent, to transfer samples between microplates and to be used sterilely.

The present invention is directed to a method of automating preparation of plates containing one or more wells with one or more layers of a semi-solid matrix.

Volume Delivery Control

As the number of wells per plate increases, or alternatively, as the volume capacity of a given well decreases, it is more important to be able to precisely control volume delivery of each reagent to each well. In addition, it is preferred that the method prepare plates with wells containing one or more pre-determined dilutions of reactants. The present method is suitable for preparing plates for use in testing compounds, and therefore, the ability to prepare wells of accurate, reproducible delivery of specified amounts of one or more reactant is important.

In one embodiment, the method automates preparation of multi-well plates with a semi-solid matrix which contains cells.

In another embodiment, the method automates preparation of multi-well plates with more than one layer of semi-solid matrix.

In another embodiment, the method automates preparation of multi-well plates with more than one layer of semi-solid matrix, but where one matrix contains cells.

In one embodiment of the invention, the assay method is used for determining the effect of a test compound on a cell in a predetermined well comprising semi-solid matrix, comprising:

(a) using a liquid handler with a first reservoir and a second reservoir to transfer a liquid semi-solid matrix from the first reservoir to a predetermined well of an assay plate;

(b) using a liquid handler to transfer cells from a second reservoir to the predetermined well of an assay plate;

(c) allowing the liquid semi-solid matrix in the predetermined well to solidify;

(d) incubating the cells in the plate for a period of time for the cells to grow into a colony;

(e) adding a predetermined amount of a test compound to the predetermined well; and

(f) determining the biological effect of the test compound on the colony.

In one embodiment of the invention, the semi-solid matrix solidifies at about room temperature. In another embodiment, the semi-solid matrix solidifies at between 10 to 45 degrees Celsius. In a preferred embodiment, the semi-solid matrix solidifies between 15 to 40 degrees Celsius. In yet another embodiment, the semi-solid matrix solidifies between 20 to 35 degrees Celsius. In a more preferred embodiment, the semi-solid matrix solidifies between 20 to 30 degrees Celsius.

In one embodiment of the invention, the semi-solid matrix comprises agar, agarose, collagen, or basement membrane. In another embodiment of the invention, the semi-solid matrix comprises agar, agarose, or collagen. In yet another embodiment of the invention, the semi-solid matrix comprises agar or agarose. In yet another embodiment of the invention, the semi-solid matrix comprises agarose. In yet another embodiment of the invention the semi-solid matrix comprises collagen or Matrigel®. In yet another embodiment of the invention the semi-solid matrix comprises Matrigel®.

In one embodiment of the invention, the semi-solid matrix is heated until it is a liquid before the liquid semi-solid matrix is added to the first reservoir. In another embodiment of the invention, the semi-solid matrix is heated until it is a liquid after the semi-solid matrix is added to the first reservoir.

In one embodiment of the invention, the semi-solid matrix is cooled until it is a liquid before the liquid semi-solid matrix is added to the first reservoir. In another embodiment of the invention, the semi-solid matrix is cooled until it is a liquid after the semi-solid matrix is added to the first reservoir.

In one embodiment of the invention, the assay method used for determining the effect of a test compound on cells comprises:

(a) using a liquid handler to transfer a liquid semi-solid matrix from a first reservoir to a predetermined well of an assay plate;

(b) using a liquid handler to transfer growth medium from a second reservior to the predetermined well of the assay plate;

(c) using a liquid handler to transfer cells from a third reservoir to the predetermined well of the assay plate;

(d) allowing the liquid semi-solid matrix in the predetermined well of the assay plate to solidify;

(e) incubating the cells in the predetermined well of the assay plate for a period of time for the cells to grow into a colony;

(f) adding a predetermined amount of a test compound to the predetermined well of the assay plate; and

(g) determining the biological effect of the test compound on the colony.

In yet another embodiment of the invention, the assay method used for determining the effect of a test compound on cells comprises:

(a) using a liquid handler to transfer a liquid semi-solid matrix from a first reservoir into a predetermined well of the assay plate;

(b) using a liquid handler to transfer growth medium from a second reservior to the predetermined well of the assay plate;

(c) using a liquid handler to transfer cells from a third reservoir to the predetermined well of the assay plate;

(d) using a liquid handler to transfer a predetermined amount of a test compound from a fourth reservoir to the predetermined well of the assay plate;

(e) allowing the liquid semi-solid matrix in the predetermined well of the assay plate to solidify;

(f) incubating the cells in the predetermined well of the assay plate for a period of time for the cells to grow to form a colony; and

(g) determining the biological effect of the test compound on the colony.

In one embodiment of the invention, the liquid handler is used to prepare dilutions of the test compound in predetermined wells in a dilution plate. In another embodiment of the invention, the liquid handler transfers a predetermined amount of test compound from a predetermine well in the dilution plate to the predetermined well of the assay plate.

In one embodiment of the invention, the assay plate comprises six or more wells. In another embodiment of the invention, the assay plate comprises twenty-four or more wells. In yet another embodiment of the invention, the assay plate comprises forty-eight or more wells. In a preferred embodiment of the invention, the assay plate comprises ninty-six or more wells. In a more preferred embodiment of the invention, the assay plate comprises 384 or more wells.

In one embodiment of the invention, the cells are capable of non-adherent growth in a semi-solid matrix. In another embodiment of the invention, the cells are normal primary cells, stem cells, or tumor cells. In another embodiment of the invention, the cells are normal primary cells. In another embodiment of the invention, the cells are stem cells. In another embodiment of the invention, the cells are tumor cells. In another embodiment of the invention, the tumor cells are breast tumor cells, ovarian tumor cells, melanoma cells, neuroblastoma cells, colon tumor cells, prostate tumor cells, large cell lung tumor cells or small cell lung tumor cells.

In one embodiment of the invention, the effect of the test compound on the colony is determined by a use of a luminometer, use of a photometer, use of scintillation, use of fluorescence, or by visual counting of colonies. In another embodiment of the invention, the effect of the test compound on the colony is determined by use of fluorescence or by use of a luminometer. In another embodiment of the invention, the effect of the test compound on the colony is determined by a viability stain. In another embodiment of the invention, the viability stain is a fluorescent probe. In another embodiment of the invention, the viability stain is Calcein AM.

In one embodiment of the invention, the liquid handler is capable of transfering a volume of a liquid from about a nanoliter to about five microliters to a predetermined well. In another embodiment of the invention, the liquid handler is capable of transfering a volume of a liquid from about one microliter to about five hundred microliter to a predetermined well.

In one embodiment of the invention, the liquid handler is capable of maintaining a predetermined temperature of a reservoir. In another embodiment of the invention, the liquid handler is capable of simultaneous transfer of a liquid to more than one well.

In another embodiment of the invention, the assay plate is a microtube.

In another embodiment of the invention, the semi-solid matrix comprises at least 25 percent agar, agarose, collagen, or basement membrane. In an alternative embodiment of the invention, the semi-solid matrix comprises at least 45 percent agar, agarose, collagen, or basement membrane. In another embodiment of the invention, the semi-solid matrix comprises at least 60 percent agar, agarose, collagen, or basement membrane. In another embodiment of the invention, the semi-solid matrix comprises at least 75 percent agar, agarose, collagen, or basement membrane.

In another alternative embodiment of the invention, the semi-solid matrix comprises at least 25 percent agarose. In another embodiment of the invention, the semi-solid matrix comprises at least 45 percent agarose. In another embodiment of the invention, the semi-solid matrix comprises at least 60 percent agarose. In another embodiment of the invention, the semi-solid matrix comprises at least 75 percent agarose.

In one embodiment of the invention, the semi-solid matrix comprises at least 25 percent agar. In another embodiment of the invention, the semi-solid matrix comprises at least 45 percent agar. In another embodiment of the invention, the semi-solid matrix comprises at least 60 percent agar. In another embodiment of the invention, the semi-solid matrix comprises at least 75 percent agar.

In one embodiment of the invention, the assay plate comprises from 50 wells to 2000 wells. In another embodiment of the invention, the assay plate comprises from 50 wells to 200 wells. In another embodiment of the invention, the assay plate comprises from 200 wells to 500 wells. In another embodiment of the invention, the assay plate comprises from 300 wells to 500 wells. In another embodiment of the invention, the assay plate comprises from 350 wells to 2000 wells.

In one embodiment of the invention, the liquid handler is an automated system.

In another embodiment of the invention, the effect of the test compound on the colony is determined by the cell viability. In another embodiment of the invention, the cell viability to determined by a fluorescent probe.

In one embodiment of the invention, the method of automating preparation of plates containing semi-solid assay matrix using a liquid handler for an assay including the addition of compound to the semi-solid matrix, comprises

using a plate with multiple wells arranged in rows and columns with each well capable of holding a predetermined liquid volume, where the liquid handler contains multiple reservoirs; wherein a first reservoir containing a liquid growth medium in first 1× concentration; wherein a second reservoir containing a liquid growth medium in second concentration 2×; wherein a third reservoir containing a liquid semi-solid matrix; wherein a fourth reservoir containing a liquid growth medium in third concentration 1.67×; wherein a fifth reservoir containing cells suspended in 1× growth medium, and wherein the first step is to perform a serial dilution of a test compound into a dilution plate wherein one well in each column is designated a starting well and the remaining wells are designated dilution wells, where the dilution wells contain growth medium, by

a. transferring a predetermined amount of 1× growth medium from the first reservoir to each dilution well in that column,

b. transferring a predetermined amount of test compound from the starting well into first dilution well,

c. mixing the contents of the first dilution well,

d. transferring the same predetermined amount of liquid from the first dilution well into a second dilution well,

e. mixing the contents of the second dilution well,

f. repeating steps b and c as required.

The method may additionally comprise a second step by preparing a second plate to mix the components of the semi-solid matrix, by

a. transferring a predetermined amount of each well from the first plate to a corresponding well in a second plate, designated a mix plate,

b. transferring a predetermined amount of 2× growth medium from the reservoir to each well in mix plate,

c. transferring a predetermined amount of semi-solid matrix from the reservoir to each well in mix plate,

d. mixing the contents of each well in the mix plate, while the semi-solid matrix is still liquid,

The method may additionally comprise a third step by preparing a first layer of semi-solid matrix in a third assay plate, by

a. transferring a predetermined amount of each well from the mix plate to a corresponding well in a third plate, designated the assay plate,

b. allowing the semi-solid matrix to solidify at the desired temperature,

The method may additionally comprise a fourth step by preparing a second layer of semi-solid matrix in a third, assay plate, by

a. transferring a predetermined amount of each well from the first plate to a corresponding well in a fourth plate, designated a second mix plate,

b. transferring a predetermined amount of 1.67× growth medium from the reservoir to each well in second mix plate,

c. transferring a predetermined amount of cells in 1× growth medium from the reservoir to each well in second mix plate,

d. transferring a predetermined amount of semi-solid matrix from the reservoir to each well in second mix plate,

e. mixing the contents of each well in the second mix plate, while the semi-solid matrix is still liquid,

f. transferring a predetermined amount of each well from second mix plate to a corresponding well in a third assay plate on top of the first layer of semi-solid matrix,

g. allowing the semi-solid matrix to solidify at the desired temperature,

In addition, the method may also include the additional step of removing the plate to incubate for predetermined period at desired temperature and conditions.

The following Examples provide additional specific teachings of the use of the present invention, but are not intended to be limitations on the uses of the present invention.

### EXAMPLE 1

### Cells Capable of Non-Adherent Growth

Cell lines useful in the instant method include the following cell lines which are capable of non-adherent growth on semi-solid matrices. The cell lines provided herein are available from commercial sources such as the American Tissue Type Collection, ATCC (Manassas, Va.). Cells may also be obtained directly from patients using well-known methods for isolation of suitable cells.

Cells lines DU-145, Colo205, NCI-H460, PC-3, HL-60, MDA-MB 435, MDA-MB231 and MCF-7 were obtained from ATCC. Cell lines transfected with green fluorescent protein (GFP) (purchased from AntiCancer, Inc., San Diego, Calif.) were designated GFP-MDA-MB435, GFP-MDA-MB231, GFP-PC-3, and GFP-Colo205.

### EXAMPLE 2

### Criteria for Selection of Liquid Handler Machine

The preferred machine for automating the soft agar assay is the 96-well Liquid Handler (Denley Wellpro, Propette, Lab Systems). The properties used to select the Wellpro are the following: the speed for delivery of reagents to the wells, ability to perform serial dilutions of almost any ratio, ability to perform serial dilutions of eight or twelve rows across, ability to deliver precise amounts of reagents to each well, ability to mix the reagents and transfer reagents from wells of different plates, flexibility to adjust the head to different heights, ability to operate the machine sterilely and ease of operation of the machine.

### EXAMPLE 3

### Steps for 96-Well Format Optimization

Each well in a 96-well plate holds 0.37 ml volume, compared to the 3.5 ml volume of each well in a traditional 24-well plate, almost a ten-fold decrease in volume. Therefore, the first step was to determine the optimal volumes of each agarose layer.

The first experiment-proportionally reduced the volume of the two layers of agarose from the standard amounts used in the traditional larger multi-well plate wells. Two thicknesses in the 96-well plate well were used: 50 uL=thin and 150 uL=thick. The top layer of agarose contained serial dilutions of 1000, 5000, 10000 and 20000 cells/top layer.

The bottom layer is approximately 0.5% to about 0.7% agarose, preferably about 0.55% to about 0.65% agarose, and the top layer is approximately 0.3% to about 0.5% agarose, preferably about 0.35% to about 0.45% agarose. The bottom layer is usually denser, since it is used to support the top layer and prevents the cells from reaching the plastic substrate. If the cells reach the plastic substrate, they form a monolayer instead of colonies. However, if the density of the top layer is too high, the cells cannot expand outward, i.e., physically push outward. In addition, if the layers are too dense, perfusion of nutrients to the colony may be retarded and the cells starve to death.

### EXAMPLE 4

### Comparison of Semi-Solid Matrices

The following example provides a comparison of two types of semi-solid matrices. The two agaroses selected for comparison were: Sea Plaque #50101 FMC BioProducts and Sigma Agarose Type VII low gelling temperature #A6560. Sea Plaque agarose is often used for soft agar assays in the standard 24-well large format. Sigma Agarose Type VII low gelling temperature #A6560 is frequently used for sequencing gels.

1×RPMI growth medium (GM) was used initially. The bottom layer consisted of 50% 1×RPMI GM and 50% 1.2% agarose (water). The GM was effectively diluted in half. In the top layer, 70% of the volume was 1×RPMI GM and cells, and 30% 1.2% agarose, resulting in a final GM concentration of 0.7% in the top layer. RPMI growth medium is commercially available in 500 ml bottle (available from Gibco BRL) and consists of about 10% fetal calf serum, about 1% glutamine and about 1% antibiotics. RPMI growth medium is replaced with IMEM growth medium for MCF-7 cells. Colony formation was compared in three cells lines in 24-well and the 96-well plates.

The thin layers (50 uL) and Sigma agarose had the best colony growth. The thick layers appeared to reduce the colony growth. In the Sigma agarose, the colony formation increased with cell number in all well sizes with the thin layers. With Sea Plaque, about 50% of the cells had formed colonies. In addition, the colonies were larger in the Sigma agarose. At the end of one week, one cell line had scorable colonies, while the other two cell lines needed further growth. By the end of two weeks, all three cell lines had scorable colonies at the highest plating density in the 96-well plates. The colony morphology was similar between the different size multiplates.

Longer incubations caused the thin layer wells to gradually dry out and reduce volume. For example, some of the wells in the 50 uL thin layer dried out during the 14-day incubation period. To prevent desiccation of the agarose during the two week incubation period, the agarose layer depth was increased to 75 uL in the 96-well plates; the 75 uL layers remained sufficiently hydrated for up to four weeks incubation at 37° C. During shorter incubation periods, the layers may be smaller.

To alleviate the lack of nutrients due to the dilution of the GM, exogenous fetal calf serum may be added to the agarose mix. The additional serum brought the FCS concentration up to approximately 25% by volume. The additional serum only slightly improved colony formation, but increased the variability in the colony size; this variability was an undesirable effect. This appears to increase colony formation of some of the less robust cell lines.

### EXAMPLE 5

### Selection of Medium Concentration

The use of 1× versus 2× medium as the concentration in the preparation of the agarose layers was compared. Experiments using 1× growth medium resulted in further dilution once the other assay components were added. If 2× medium concentration was used, the growth medium could be diluted to 1× final concentration. Colony formation was generally better in the 2× medium as indicated below.

Table 1 provides a comparison of 1× Growth Medium (1×GM) versus 2× Growth Medium (2×GM) on GFP-COLO205 Cells (AntiCancer) labeled with Calcein AM (Molecular Probes, Glycine, N,N′-[[3′,6′-bis(acetyloxy)-3-oxospiro[isobenzofuran-1(3H),9′-[9H]xanthene]-2′,7′-diyl]bis(methylene)]bis[N-[2-[acetyloxy)methoxy]-2-oxoethyl]]-, bis[(acetyloxy)methyl] ester).

TABLE 1

| | 1X GM | | | 2X GM | | |
|---|---|---|---|---|---|---|
| | Fluorescent | Std | Std | Fluorescent | Std | Std |
| Cells/ml | Unit | Dev | Error | Unit | Dev | Error |
| 781 | 319 | 53 | 8.8 | 394 | 52 | 8.7 |
| 1563 | 295 | 48 | 8 | 414 | 52 | 8.7 |
| 3126 | 306 | 56 | 9 | 435 | 68 | 11.3 |
| 6250 | 312 | 61 | 10 | 519 | 81 | 13.5 |
| 12500 | 339 | 69 | 11.5 | 682 | 108 | 18 |
| 25000 | 395 | 70 | 12 | 877 | 123 | 20.5 |
| 50000 | 887 | 104 | 17 | 1328 | 246 | 41 |

The medium concentrations needed for the two layers are as follows. 2× growth medium is used for the bottom layer and 1.67× is used in the top layer. 1.67×=2× growth medium+100 ml sterile water. 2× growth medium=2×RPMI growth medium (500 ml) without phenol red, 100 ml FCS, 10 ml glutamine and 10 ml antibiotics. 2×media are available commercially.

### EXAMPLE 6

### Automated Method of Preparing Plates using a Non-Heated Liquid Handler

The following demonstrates the method of preparing plates with semi-solid matrices using a non-heated Liquid Handler, specifically using a Denley Wellpro Liquid Handler (Propette™) machine, which can fill a 96-well plate, perform serial dilutions and plate-to-plate transfers.

The Propette™ machine can be used to set up 96-well plates of soft agar. One concern in automating the assay is mechanical injury of the cells during handling. Several parameters need to be taken into account when using the Propette™ machine. For example, number of mixes, speed of action, number of cells, should be tested for each cell line used.

Speed

The speed at which the machine can pour the reagents into the individual wells is important because the agarose solidifies quickly at room temperature. A slow speed should cause fewer air bubbles to form on the plates, but risks the agarose solidifying before it is poured. A faster speed might cause more air bubbles to form in the wells, but should prevent the agarose from solidifying too soon.

The effect on air bubble formation was determined on cell plating at the three speeds, indicated as slow, medium and rapid settings. The plating speed will vary between models and different manufacturers. Air bubbles in the agarose formed at each of speeds. However, air bubbles dispersed when the plates were subsequently incubated overnight in a staggered position, as opposed to vertical stacking. As a result, the fast speed was selected, since it ensured finishing the plating before the agarose solidified. Speed would be less important if the machine contains a means to keep the agarose or plates warm during the dispersing process, i.e., contains a heating element to keep the agarose liquid during the plating.

Premixing of the Tumor Cells and Agarose

The cells, agarose and optionally compound should be mixed before being poured on to the bottom layer. If the cells were mixed in the top layer agarose after layering, they did not disperse throughout the top layer, which resulted in a cell monolayer at the agarose layer interface. A single mix prior to plating was also not sufficient to disperse the cells. Sufficient cell dispersion was obtained by a few mixes, such as two or three, which sufficiently dispersed the cells evenly throughout the agarose.

To reduce plating time, the top layer of agarose, cells and optionally compound, were mixed only twice before being transferred from the mix plates to the culture plates. The Propette™'S speed is sufficient to mix the top layer components and transfer the wells to the culture plate before the agarose of the last row solidifies.

Cell Density

A series of cell density experiments were set up to determine the optimal cell density. A higher density of about 50,000 cells/ml final concentration (working concentration 10×=500,000), was determined to be a good density for the range of cell lines being used. This is a higher density than used in the traditional soft agar assay. However, it is thought that the mechanical handling from the automation may damage some of the cells. In addition, since the colonies were not scored visually, the colonies can be closer in the 96-well plate. A higher density of cells also increases the overall fluorescence of the well.

It was determined that when plating with the test compound, the individual components, i.e., agarose, cells and compound, were more evenly distributed if premixed in a separate plate before being transferred to the 96-well plate. The test compounds were made 10× in RPMI GM, which was diluted to a 1× compound concentration in the mixing plate. The final mixing plate volume of 150 uL per well was sufficient for consistent plate-to-plate dispersal of 75 uL per layer.

Satisfactory results were obtained with the following combinations:

TABLE 2
Including the test compound in the semi-solid matrix with the cells.

| Bottom Layer | Top Layer |
|---|---|
| 15 uL 10X test compound (prepared in | 15 uL 10X test compound |
| 1X medium) | |
| 60 uL 2X medium | 75 uL 1.67X medium |
| 75 uL agarose (1.2% stock in water) | 15 uL of 500,000 cells/ml |
| | 45 uL agarose |

TABLE 3
Allowing colony formation prior to addition of test compound.

| Bottom Layer | Top Layer |
|---|---|
| 75 uL 2X medium | 75 uL 1.67X medium |
| 75 uL agarose (1.2% stock in water) | 15 uL 1X medium |
| | 15 uL of 500,000 cells/ml |
| | 45 uL agarose |

### Example 7

### Method for Automated Quantification

For optimization of automatic quantification method several staining techniques were compared. Specifically, using GFP-cell lines, MTT or calcein AM staining were compared.

For the MTT staining, the following protocol was used:

25 μL of a 10 mg/ml stock of MTT (3-[4,5-dimethyl-2-yl]-2,5-diphenyltetrazolium bromide; Thiazolyl blue) (Sigma) in Hank's Balanced Salt Solution (HBSS) is added to each well and incubated for four hours at 37 deg. C. After incubation, 200 μL dimethyl sulfoxide (DMSO) is added to each well and the plates read on a spectrophotometer at 570 nm. However, no counts could be determined with a SpectraMax 250 plate reader. For the calcein AM staining, the following protocol was used:

25 μL of 10 μM calcein AM (in HBSS) is added to each well and allowed to incubate at 37 deg. C. for at least one hour. The plate is then read in a Cytofluor 4000 (3 counts/well) with a gain of 40. The fluorescence for calcein AM (after cleavage in live cells) is excitation=485+20 nm and emission=530+20 nm.

The GFP-cells' fluorescence generally produced a weak signal, and was insufficient to be read by the CytoFluor or other fluorescent plate readers. In addition, the cells are less robust-and more sensitive to the test compounds than the parent cell lines. The MTT results were more variable than the calcein AM. The calcein AM results were more consistent, and therefore, this staining method was preferred over the other methods.

One advantage of using the viability dye as opposed to the usual method of visual quantification, is that only living cells are counted. Dead or dying cells in the colony remain held in place by the agarose. Visual counting method cannot distinguish these cells and might result in dead colonies being counted as alive. One variation of the soft agar assay is to add test compound to test colonies after colony formation. This method should be more representative of an in vivo tumor as opposed to the usual method where the compound is present when the cells are plated, resulting in individual cells being killed.

For analysis, the data provided by the CytoFluor are analyzed in EXCEL. The DMSO control wells are averaged and the standard deviation determined. Each compound well is then divided by the control average×100 for % control. The triplicate wells are averaged and the standard deviation determined. There is some background fluorescence and the colonies treated after formation are not eliminated by the single two-week treatment. The background can be subtracted from each well. Statistical programs such as PRISM could also be used for the graphing and IC₅₀ determination.

Verification

Several compounds were tested in dose response curves, including flavopiridol ((cis-(-)-5,7-Dihydroxy-2-(2-chlorophenyl)-8-[4-(3-hydroxy-1-methyl)piperidinyl]-4H-1-benzopyran-4-one hydrochloride dihydrate). As a comparison, the same compounds were tested in the traditional hand-pipetted, 24-well plates, visually counted method. The only difference was that the soft agar assay used a 500 μL agarose layer in the 24-well plate and 75 uL/layer in the 96-well system, using the Sigma agarose.

The two methods produced similar results, i.e. IC₅₀s for the compounds, see Table 4.

TABLE 4
Comparison of 24- versus 96-well Multi-well Trays.
Calcein AM stained method

| | IC₅₀ (μM) with | IC₅₀s (μM) with |
|---|---|---|
| | Flavopiridol | Flavopiridol |
| Treatment Schedule | 24-well | 96-well |
| Cells plated with 0.09 μM | 0.08, 0.15 | 0.15 |
| Flavopiridol | | |
| 0.4 μM Flavopiridol added | 0.4, 0.4 | 0.6 |
| immediately after plating | | |
| Flavopiridol added to one week | 1.5, 4 | 1 |
| colonies | | |

The 24-well plates were also counted visually, Table 5., and the IC₅₀s determined, Table 6.

TABLE 5
Visual Counting to Determine Numbers of Colonies in 24-well Tray

| | | | Flavopiridol | | | Flavopiridol | |
|---|---|---|---|---|---|---|---|
| | Plated With | | Added Immediately | | | Added to One- | |
| | Flavopiridol | | After Plating | | | Week Colonies | |
| μM | Average | Std | Average | Std | μM | Average | Std |
| Flavo. | # | Dev | # | Dev | Flavo. | # | Dev |
| 0 | 31 | 3.5 | 27 | 9 | 0 | 36.0 | 5.7 |
| 0.00032 | 27 | 4.2 | 30 | 10.4 | 0.0032 | 34.0 | 9.7 |
| 0.0016 | 25 | 10.5 | 30 | 2.6 | 0.016 | 35.0 | 6.8 |
| 0.008 | 22 | 5.5 | 32 | 7.6 | 0.08 | 32.0 | 6.9 |
| 0.04 | 25 | 1.5 | 26 | 5 | 0.4 | 33 | 1.5 |
| 0.2 | 0 | 0 | 27 | 5.9 | 2 | 25 | 8.1 |
| 1 | 0 | 0 | 0 | 0 | 10 | 32 | 7.2 |
| 5 | 0 | 0 | 0 | 0 | 50 | 20 | 3.1 |

Note:
the colonies in the flavopiridol treated wells in which the compound was added to the one-week colonies, even though still present, appeared dead.

TABLE 6
IC₅₀s Determined from Colony Counting

| | |
|---|---|
| Plated with Flavo: | 0.09 uM |
| Added immediately after plating: | 0.4 uM |
| Added to 1-week | did not reach 50% control. |

It is interesting to note Sigma agarose, which gels sufficiently when used in the 96-well format sometimes fails to gel consistently in the 24-well plate and maintain its integrity, especially when the compounds are added in growth medium after colony formation.

Table 7. provides the IC₅₀ in micromolar values obtained using flavopiridol on already formed colonies of a number of different cell lines (one week colonies). Flavopiridol is added to colonies formed after 7 days of incubation at 37 deg. F. This method of adding compounds to already formed colonies may be more predictive for the compound's effectiveness against a tumor in vivo.

TABLE 7
Flavopiridol IC₅₀ (uM) on Formed Colonies

| Cell Lines | Average ± Standard Error |
|---|---|
| Colo 205 | 1.4 ± 0.06 |
| HL-60 | 1.6 ± 0.15 |
| NCI-H460 | 2.4 ± 0.1 |
| MDA-MB435 | 2.2 ± 0.09 |
| PC-3 | 1.7 ± 0.04 |

### SUMMARY OF THE INVENTION

The invention concerns an automated method for performing biological assays and an improved liquid handling machine for automatically transferring liquid between a plurality of liquid containing wells to prepare culture trays containing semi-solid matrices for use in assays. Preferably, the machine has a head with a plunger assembly mounted on the head, the plunger assembly including a plurality of pipettes having depending ends for receiving tips. A plurality of plungers are respectively disposed within the pipettes, the plungers being movable coaxially within the pipettes to vary their internal volumes.

The machine also has a tip ejector for removing tips disposed on the depending ends of the pipettes. A support, preferably in the form of an elongated table, is mounted beneath the head, the support having at least a first work station being adapted to accommodate a tray having pipette tip-receiving receptacles, and a second and a third work station, each being adapted to accommodate a respective tray of the liquid containing wells. The support and the head are movable relatively to one another to place the pipettes in registry with any of the tips in the tip-receiving receptacles and any of the wells in registry with the pipettes.

A motion controller, preferably a microprocessor, is used for controlling the relative motion between the head and the support, as well as the motion of the plungers to effect transfer of liquid between the liquid containing wells. The motion controller also controls the action of the tip ejector to replace tips on the ends of the pipettes with other tips disposed in at least some of the pipette tip-receiving receptacles between predetermined liquid transfer steps.

The machine is improved in that the support has a plurality of temperature control elements for independently controlling the temperature at each of the second and third work stations to maintain the liquid in the liquid containing wells at a predetermined temperature. The support also has a sensor at each of the second and third work stations for sensing the temperature, and a temperature controller, preferably a microprocessor, for controlling the temperature control elements and the sensors.

Preferably, the temperature control elements comprise independent heaters and cooling devices for heating or cooling the table at each of the second and third work stations. The machine also has an insulating strip for thermally isolating the second and third work stations from one another, the insulating strip being interposed between the second and third work stations to prevent heat transfer and allow the stations to be maintained at different temperatures by the respective temperature control elements.

Practical temperature control elements include electrical resistive heaters, Peltier effect modules and heat pumps or other independent heating or refrigeration devices which can pump a working fluid through a passageway arranged in the support table beneath the second and third work stations to effect heating and cooling of the stations to maintain the assay components at the desired temperatures.

The improved liquid handling machine can also be combined with machines for scoring the assay results, for example, by fluorescence, spectrophotometric or other techniques.

### BRIEF DESCRIPTION OF THE DRAWINGS

FIG. 1 shows a partial perspective view of an improved liquid handling machine according to the invention;

FIG. 2 shows a partial side view of the liquid handling machine shown in FIG. 1;

FIG. 3 is a partial cross-sectional side view of a detail of the machine shown in FIG. 2 on an enlarged scale;

FIG. 4 is a partial cross-sectional side view of a detail of the machine shown in FIG. 3 on an enlarged scale;

FIG. 5 is a partial cross-sectional side view of a detail of the machine shown in FIG. 3 on an enlarged scale;

FIG. 6 is a partial cross-sectional top view of the machine shown in FIG. 1;

FIG. 7 is a top view of a detail of the machine shown in FIG. 6;

FIG. 8 is an end view of the machine shown in FIG. 1;

FIG. 9 is a partial top view of a detail of the machine shown in FIG. 8;

FIG. 10 is a partial cross-sectional view taken along line 10-10 of FIG. 8;

FIG. 11 is a partial cross-sectional view taken along line 11-11 of FIG. 8;

FIG. 12 is a partial perspective view of a detail of the machine shown in FIG. 1;

FIG. 13 is a side view of an improved liquid handling machine according to the invention; and

FIG. 14 is a side view of an improved liquid handling machine according to the invention incorporating a device for automated evaluation of biological assay results.

### DETAILED DESCRIPTION OF THE PREFERRED EMBODIMENT

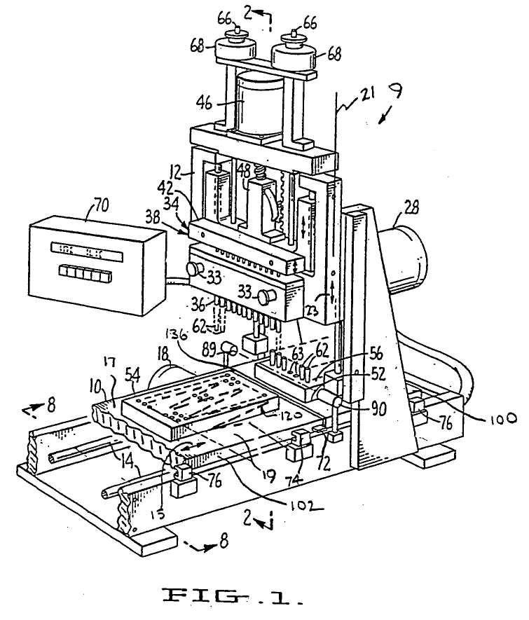

FIGS. 1, 2 and 8 show an improved liquid handling machine 9 capable of regulating the temperature of assay compounds in the automated and semi-automated preparation of culture trays for biological assays such as soft agar assays for culturing tumorigenic cells, stem cells and/or bone marrow cells. Machine 9 includes two main movable parts, a horizontally translatable table 10 and a vertically translatable head 12. As best illustrated in FIG. 2, the table 10 is mounted for horizontal translation on hardened guide rods 14 by means of slide bearings 16. The table is thus movable on guide rods 14 along a path denoted by arrow 15 in a horizontal plane 17 preferably coplanar with the surface 19 of table 10. Translation of the table is provided by a stepper motor 18 through a pinion 20 connected to the motor and a rack 22 mounted on the underside of the table. The head 12 is mounted on guide rods 24 by means of slide bearings 26 and is thus capable of translational motion along an axis 21 substantially perpendicular to the surface 19 of table 10 as denoted by arrow 23. Translation of the head is provided by a stepper motor 28 through a pinion 30 and a rack 32.

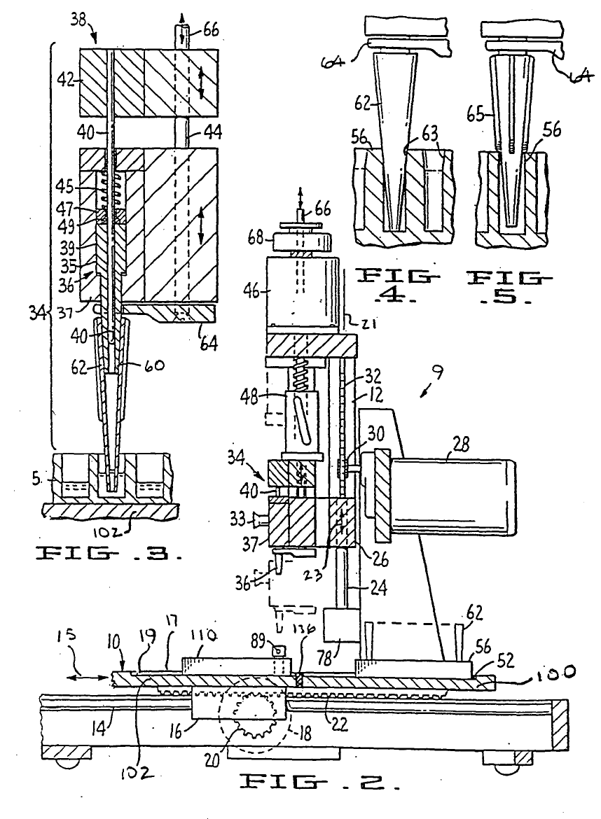

As shown in FIGS. 2 and 8 the head 12 supports a pipette and plunger assembly 34. This assembly includes a plurality of pipettes 36 that are arranged in a row transverse to the axis of translation of the table 10. The pipettes are removably attached to the head by means of a mounting block 37 and connecting pins 33 so that different pipette assemblies having more or fewer pipettes can be readily interchanged. The pipettes move with the head 12. As shown in detail in FIG. 3, a plunger mechanism 38 is mounted on the head for vertical movement relative to the pipettes. The plunger mechanism includes a series of plunger rods 40, one being disposed respectively within each pipette 36. All of the rods are mounted on a common actuator bar 42 for concurrent vertical movement. The bar 42 is translated along guide rods 44 by means of a stepper motor 46 and a lead screw drive mechanism 48 (see FIG. 2). As best illustrated in FIG. 3, translation of the plunger rods 40 relative to the pipettes 36 changes the internal volumes of the pipettes, causing fluid in which the pipette tips are immersed to be aspirated into or expelled from them, thus, allowing the pipettes to effect fluid transfer in the assay procedure as described below. An air tight seal is provided between each rod and the top of its associated pipette by means of an O-ring 49, held by grommet 47 and compliance spring 45.

Referring again to FIG. 3, the bottom end 60 of each pipette 36 is tapered on its exterior surface so as to receive and frictionally engage the inner surface of a disposable pipette tip 62. Tip 62 is preferably made of a non-wettable polypropylene material for reasons explained below. As shown in FIGS. 1, 2 and 4, the tips 62 are stored in rows of receptacles 63 in a tip tray 56 which is positionable on table 10. To attach the pipette tips 62 to the pipettes 36, table 10 is moved by stepper motor 18 to bring one row of tips into registry with the pipettes and the head 12 is lowered by the stepper motor 28 to frictionally engage the tapered portion of pipettes 36 with the pipette tips. As shown in FIG. 3, each pipette 36 includes a piston section 39 which is reciprocally mounted in a cylinder 35 formed in mounting block 37. Pipettes 36 are thereby restrained vertically by springs 45 so that when disposable pipette tips 62 are temporarily attached to the ends of the pipettes during the tip loading step pipettes 36 can slide vertically in block 37 against compliance springs 45. This allows all pipettes to reliably pick up tips of slightly different dimensions and to assure that the open ends of tips 62 are at the same elevation relative to table 10 and a culture tray 54 (described below) positioned on the table.

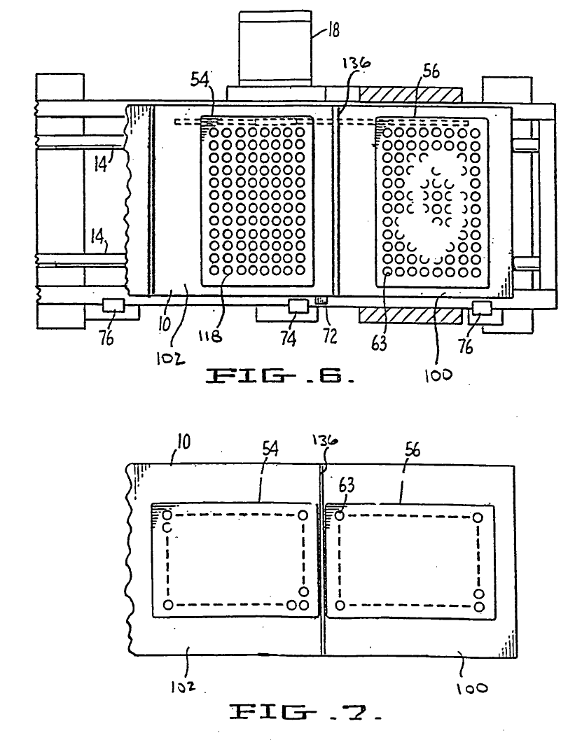

As indicated in FIG. 3, the volume of each tip 62 is a substantial portion of the total volume of the cylinder formed by pipette 36 and the interior volume of the tip. As best seen in FIGS. 4 and 5, support of each tip 62 in receptacle 63 of tray 56 is either by end support as in FIG. 4 or on ends of the bottom flutes 65 formed on the exterior of tips 62. The wall of receptacles 63 are arranged to center tip 62 for engagement with tapered ends 60 of pipettes 36.

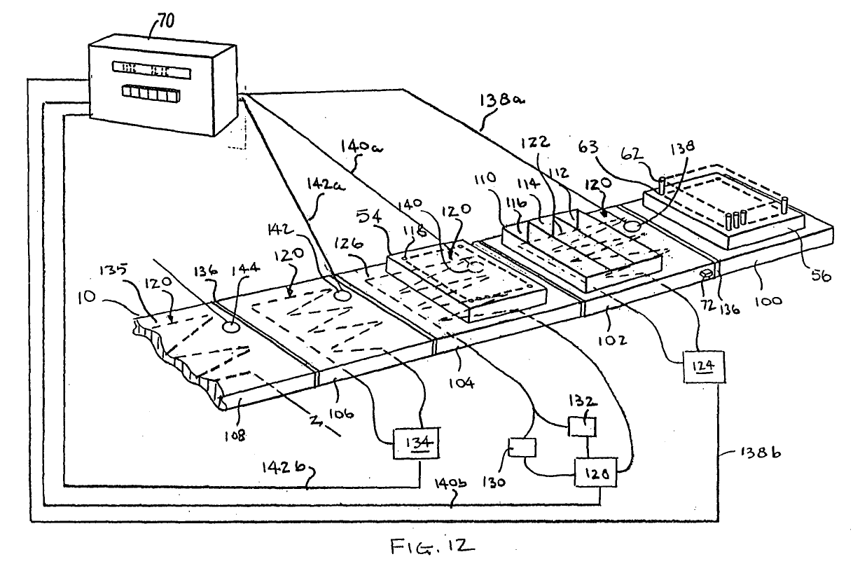

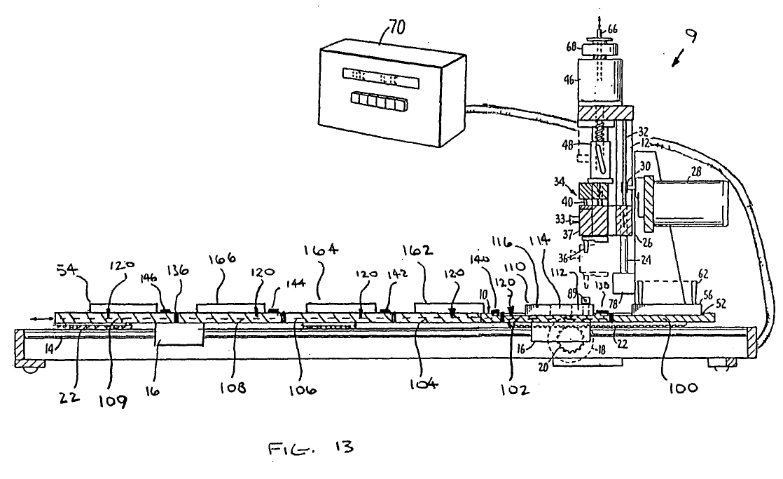

The removal of the tips 62 from the pipettes is accomplished with a tip ejector which includes a comb-like tip ejector plate 64 that is best illustrated in FIG. 11. The plate has recesses that accommodate the pipettes, and its teeth surround a substantial portion, e.g., 180°, of the exterior circumference of each pipette. The plate 64 is connected to and supported by a pair of vertically translatable rods 66 mounted on the head 12 (see FIG. 10). These rods are translated by means of a pair of solenoids 68 (see FIGS. 1 and 9) mounted on the top of the head. When the solenoids 68 are deactuated, the tip ejector plate 64 is maintained in the upper position illustrated in FIG. 5. Actuation of the solenoids moves the plate vertically downward, to push the tips 62 down and release them from their frictional engagement with the ends of the pipettes 36.

As best shown in FIG. 12, table 10 is divided into a plurality of work stations 100, 102, 104, 106, 108 and so forth, the work stations being spaced on table 10 along its path of motion 15. The work stations accommodate the various components necessary to perform the automated soft agar assay according to the invention. The detailed description of the table and the work stations below is for the preferred embodiment, it being understood that variations in the construction and organization of the table and the various components are included within the scope of the invention.

In performing the automated soft agar assay, pipettes 36 on head 12 are used to repeatedly transfer precisely measured amounts of different fluids, such as agar, diluent, liquid growth medium containing viable cells, and the compound whose effect on the cells is to be assayed, between various reservoirs and wells (described below) positioned on table 10. The work stations 100 et seq are preferably arranged linearly, one adjacent to another along the length of the table 10, each work station being positionable as necessary beneath the head 12 by the action of stepper motor 18 to allow the pipettes 36 to engage the various reservoirs and wells located at each station and effect the transfer of the various fluids necessary for the assay.

Preferably, one station, such as station 100, accommodates a tray 56 which holds a plurality of the disposable pipette tips 62 in an array of receptacles 63 (see also FIGS. 1 and 4). As described above, disposable pipette tips 62 are temporarily positioned on the ends of pipettes 36 (see FIG. 3) and are used to avoid cross contamination when multiple assay samples of different compounds or compound concentrations are being prepared, a new tip being used to transfer fluid for each different concentration or compound. Station 100 acts as storage location from which pipette tips 62 can be drawn and positioned on the ends of pipettes 36 as required by translating table 10 to position a particular row of tips in registration beneath the pipettes 36 and lowering head 12 to engage the pipettes with the tips. Further details of this operation are described below.

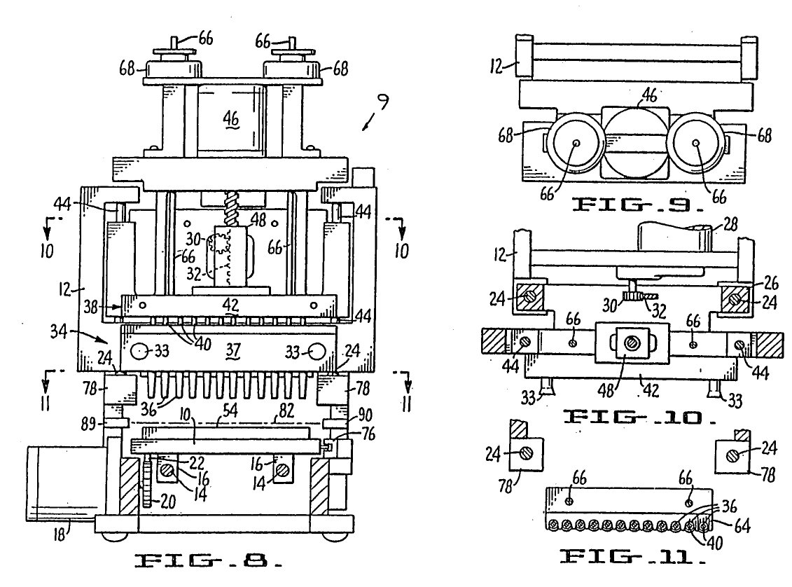

As seen in FIGS. 6 and 7, tray 56 can bc arranged on table 10 in either of two configurations, varying the number of receptacles 63 arranged transversely to the direction of motion of table 10. FIG. 6 shows tray 56 having 96 receptacles arranged with 12 columns of 8 rows transverse to the table 10. By rotating the tray 90° on the table as shown in FIG. 7, the receptacles 63 are presented to the pipettes in 8 columns of 12 rows. The size and orientation of tray 56, as well as the number of pipettes 36 in head 12, are matched to the type and orientation of the culture trays (described below) used in the assay.

Referring again to FIG. 12, another station on table 10, such as station 102, is used to hold a reservoir 110. Reservoir 110 can take on any practical form but is preferably an open-topped trough having multiple compartments 112, 114 and 116, for example, which are used to hold the various fluid components such as the agar, cell growth medium, viable cells, diluent and compound (or vehicle) used to prepare trays suitable for soft agar assays. Three compartments are illustrated in FIG. 12, it being understood that this is by way of example only and not meant to limit the configuration of reservoir 110 in any way.

Other stations, such as 104, 106 and so forth, preferably accommodate other components used in the assay such as mixing trays which are used to prepare a mixture of the viable cells with the growth medium and the compound, compound trays which are used to perform serial dilution of the compounds to be assayed, and culture trays which are used to hold a multiplicity of samples comprising a mixture of the agar, growth medium, cells and compound to be assayed. As seen in FIG. 6, the culture trays 54 preferably have an array of wells 118, each of which is used to hold a sample for the assay.

The configuration of the culture trays is not limited to any particular arrangement of wells, but standard industry trays having 6, 12, 24, 48, 96, 256 or as many as 1,530 wells have evolved. The trays are generally a standard size, which means that the well size decreases as the number of wells in a tray increases.

The trays 54 permit the samples to be arranged in ordered groups as a function of the compound being evaluated and its particular concentration. For example, shown in FIG. 6 is a standard culture tray having wells 118 arranged in 12 columns of 8 rows for a total of 96 wells. This tray configuration is useful, for example, in the evaluation of inhibitory compounds in oncological studies because it permits three different inhibitory compounds to be evaluated in triplicate at eight different concentrations against a control culture of cells which are not subjected to the compound but do contain vehicle (discussed below). Once incubated and scored (evaluated for cell growth), the samples arranged in the tray provide the data necessary to perform statistical analyses and determine the optimum concentration of each compound for inhibiting cell growth. To generate such information, one set of three columns of wells contains the control samples of cells which are not subjected to any inhibitory compound, and the remaining three sets of three columns of wells 118 each contain an inhibitory compound and each of the eight rows holds a different concentration of the respective compound. The information derived from these types of assays are used particularly in oncological studies but would also be useful in assays related to stem cells and bone marrow related compounds.

The automated method and apparatus according to the invention allow complex assays of several compounds and many different concentrations to be efficiently performed in a fraction of the time that it would take a laboratory technician to perform manually. However, the steps in the procedure, for example, transferring agar from reservoir 110 to wells 118, transferring growth medium from the reservoir to the wells, transferring viable cells and the compound in various concentrations to the wells still takes some time to accomplish. The procedure is usually performed at room temperature (typically about 25° C.) which is below the temperature at which the agar solidifies (typically about 37° C.). Thus, unless steps are taken to control the temperature of the agar it may solidify in the reservoir before it can be transferred to the wells 118 in the culture tray 54, or the agar may solidify in the wells 118 before growth medium, viable cells or the compound can be transferred and mixed with the agar to form the sample to be assayed. Temperature control becomes increasingly critical as the number of wells in a tray increases because the volume of each well decreases (the overall dimensions of the tray remaining constant regardless of the number of wells). The smaller the well volume the less time the agar takes to solidify at room temperature, thus the more wells there are per tray the less time there is to perform all of the steps necessary to prepare that tray for the assay if the temperature of the tray is not controlled. Trays having 96 wells or more cannot practically be used in the soft agar assay without temperature control because the time required for agar solidification is so short. With the industry trend going to ever larger numbers of smaller volume wells to save material costs and speed the assay process, it can be expected that temperature control will be of increasing importance as time goes on.

To prevent premature solidification of the agar, work stations such as 102 through 108 are provided with temperature control elements 120 for independently controlling the temperature at each of the work stations to maintain the liquid in the reservoir 110 or trays 54 positioned on the table 10 at a desired temperature. The temperature control elements include independent heaters for heating the table at each work station. The temperature control elements work in cooperation with independent sensors for sensing the temperature at each work station and a temperature controller for controlling the temperature control elements and sensors. The temperature control elements also include cooling devices for cooling each station, the cooling devices also being controlled by the temperature controller.

Shown schematically in FIG. 12, the temperature control elements may assume any practical form, for example, simple electrical resistive heating elements such as 122 shown for station 102. Such heating elements are mounted on or beneath the surface of the table 10 at each station and are powered by a power supply 124.

The temperature control elements alternately comprise interconnected passageways 126 within the table 10, as seen at station 104. A fluid is heated or cooled and circulated through the passageways 126 to heat or cool the trays or reservoir positioned on the tray. As shown for station 104, the fluid could be a liquid, such as water, glycol or a mixture thereof which is pumped from a reservoir 128 and then circulated through the passageways 126, the liquid being heated or cooled by passage through an external heater 130 or refrigerator unit 132 as required to establish and maintain trays 54 at a desired temperature above the solidification temperature of the agar. The fluid may be a gas, for example, heated or cooled air, or the working fluid, such as ammonia or Freon, from a heat pump 134 as shown for station 106. The working fluid is cooled or heated as required by the heat pump to maintain the desired temperature of the station, keeping the agar liquid or allowing it to solidify as desired. Yet another example of a practical temperature control element is a Peltier module 135 shown at station 108. The Peltier module is an electrically powered solid state device which can be used to either heat or cool the trays at the work stations. Operation of the module is based upon the Peltier effect, a thermoelectric phenomenon which occurs when a current is passed through a junction of two dissimilar metals or a metal-semiconductor junction. The junction is either warmed or cooled according to the direction of current flow through the junction. The Peltier effect is reversible, i.e., reversing the current causes the cooling junction to become hot and the hot junction to cool. When connected to an electric power supply (not shown) Peltier module 135 can be used as a heating or cooling device to either heat or cool work station 108. While a different temperature control element for heating and cooling each station is shown, it is understood that this is for purposes of example and illustration only and does not imply-that different elements would actually be used at each station in a practical machine.

Preferably, each work station has its own dedicated temperature control elements 120 which is independently operated for each station, allowing each station, and the tray or reservoir positioned thereon, to be maintained at a different temperature from another station. To enable such independent control of the station temperature, it is necessary to thermally isolate each station from its neighboring station. This is accomplished by interposing an insulating strip 136 at the interface between stations, as seen at FIG. 12. The insulating strip will block conductive heat transfer between adjacent stations allowing them to be maintained at different temperatures by their respective temperature control elements. The insulation material comprises glass in sheet form, glass fiber, ceramic, plastic, rubber wood and so forth to effect the thermal isolation of the stations.

The temperature control elements work in cooperation with independent temperature sensors such as illustrated by items 138, 140 and 142 located at each station. The temperature sensors are preferably transducers which generate an electrical signal in response to the temperature of the station on which they are mounted. This signal is provided by way of a respective connecting wire (138a, 140a or 142a) to a temperature controller for controlling the temperature control elements and sensors, such as a microprocessor 70, which interprets the signal and then sends control signals to the temperature control elements 120 via other connecting wires 138b, 140b and 142b to heat or cool the station as required to maintain the station, and the tray or reservoir positioned thereon, at a desired temperature which has been pre-programmed into the temperature controller. For example, if, for temperature control elements 120 comprising resistive heating elements 122 powered by power supply 124, the temperature sensor 138 signals (via connecting wire 138a) to the microprocessor 70 that the temperature of station 102 has slipped below a set temperature (typically about 42° C., the preferred temperature of the agar), meaning the agar in the reservoir 110 will begin to solidify, then the microprocessor signals to the power supply 124 (via wire 138b) to send electrical current through the resistive heating element 122 until the sensor 138 generates a signal indicating that the temperature of station 102 is again at the desired temperature which will keep the agar in a liquid state.

Similarly, for the temperature control element 120 at station 104 which comprises a series of passageways 126 through the table 10 through which a heated or cooled liquid or gas is circulated, a signal from temperature sensor 140 indicating that the temperature of station 104 is below the desired level will-result in the microprocessor commanding liquid (or gas) to be pumped from the reservoir through the heater 130 and further through the passageways 126, thereby raising the temperature of the table at station 104 to the desired level.

In a third example, for station 106 where the heating and cooling elements comprises passageways 126 through which the working fluid of the heat pump 134 can circulate, a signal from temperature sensor 142 to the microprocessor that the temperature of station 106 is too hot such that it might kill viable cells held in a tray on that station, will result in the microprocessor commanding the heat pump to circulate cooling fluid, such as Freon or ammonia, through the passageways in the manner of a refrigerator to cool the tray and maintain the temperature of the station at the desired level.

The operation of each of the stepper motors 18, 28 and 46 and the solenoids 68 is controlled by a motion controller, preferably microprocessor 70. Basically, the microprocessor 70 functions as a pulse generator to control the sequence of operations of each of these elements, and thus the interrelated movements of the table 10, the head 12, the plunger assembly 34 and the tip ejector plate 64 to effect for example serial dilution of a sample in the tray 54 at a work station. Since the stepper motors provide a predetermined amount of rotation in response to each actuating pulse applied thereto, accurate positioning of the movable elements can be obtained through appropriate control of the number of actuating pulses supplied by the microprocessor.

In addition to controlling these various movable elements, the microprocessor 70 also monitors their movement through appropriately positioned sensors. For example, as shown in FIGS. 1 and 6, a sensor arrangement for the table 10 includes a blade 72 that is attached to and extends from the side of the table, and a Hall-effect sensor 74 that detects when the blade 72, and hence the table 10, passes through a predetermined reference point in its translation. Each time the table passes through this point, the Hall-effect sensor 74 sends a signal to the microprocessor 70 that enables the microprocessor to update information relating to the table's position. Thus, if the stepper motor 18 should miss an actuating pulse during translation of the table, or if the pulse count stored within the microprocessor 70 should not coincide with the position of the table, the error will not be carried over to successive cycles of operation.

In addition to the reference sensor 74, a pair of limit sensors 76 is disposed at the respective ends of the path of travel of the table. A signal sent by these sensors indicates that the table is nearing the end of its travel and provides an indication to the microprocessor 70 to interrupt the supply of power to the stepper motor 18 or take some other such corrective action. Similar sensor arrangements are provided to monitor the movement of the head 12 and the plunger bar 42.

A sensor is also provided on the machine to detect whether all of the tips in a row of the tray 56 have been picked up by the pipette assembly. Referring to FIG. 8, this sensor preferably includes an electrical-optical mechanism comprising an LED 89 or similar such light emitting device on one side of the table and a photoelectric element 90 on the other side of the table. The two elements are aligned with the row of pipettes 36. When one or more tips 62 are present within the row of wells 63 registered with the sensor, the light beam 82 from the LED will be broken and will not reach the photoelectric element 90. However, if all of the tips in a row are successfully picked up by the pipette assembly, the beam will extend across the tray 56 and be detected by the photoelectric element. By proper positioning LED 89 and photoelectric element 90, possible pick up of tray 56 itself, as by friction between tips 62 and wells in tray 56, can also be detected.

Description of Machine Operation for Serial Dilution

The serial dilution operation is an important function performed by a liquid handling machine. In performing this operation, the improved liquid handling machine functions to pick up a row of tips in the tray 56, insert them in one row of wells in the culture tray 54, extract some of the liquid sample from these wells, inject the tips into a diluent in the next successive row of wells, oscillate the plungers to mix the liquid, position the tips to expel all liquid and then return the tips to the tray 56. This operation is set forth in greater detail with reference to the following example of a program that can be used by the microprocessor to effect a serial dilution process.

| Step | Command | Action |
|---|---|---|
| 001 | Table to position M | Bring row M of tray 56 under pipettes |
| 002 | Head down | Load tips |
| 003 | Head up | Pick up tips |
| 004 | Detect for complete | Yes: Go to 005 |
| | tip pick-up | No: Go to 002 |
| 005 | Table to position N | Bring row N of tray 54 under tips |
| 006 | Head down | Insert tips in wells |
| 007 | Plunger up | Aspirate sample into pipettes |
| 008 | Head up | Remove tips from wells |
| 009 | Table to position N + 1 | Bring next row of tray 54 under tips |
| 010 | Head down | Insert tips in wells |
| 011 | Oscillate plunger | Mix sample and dilutant |
| 012 | Head up | Tips just out of liquid in |
| | partially | wells |
| 013 | Plunger down | Expel sample from pipette |
| 014 | Head up | Above meniscus |
| | slightly further | |
| 015 | Plunger down beyond | Expel all of sample and some |
| | initial point | air |
| 016 | Head to top | |
| | position | |
| 017 | Plunger up to initial | |
| | point | |
| 018 | Table to position M | Bring empty row of tray 56 under tips |
| 019 | Head down | Insert tips in receptacles |
| 020 | Tip ejector down | Release tips |
| 021 | Tip ejector up | |
| 022 | Detect for complete | Yes: Go to 023 |
| | tip ejection | No: Go to 020 |
| 023 | Head up | |
| 024 | M = M + 1, N = N + 1 | |
| 025 | Table to position M | |
| 026 | Go to 002 | |

The cycle is optionally repeated a number of times equal to the number of dilutions to be carried out. During any given cycle, steps 001-004 and 018-022 can be deleted if changing of the tips is not required.

Prior to the initiation of a serial dilution operation, the microprocessor 70 can be programmed with the volume of liquid that is to be transferred during each cycle of the process. This amount determines the extent to which the plunger rods 40 are raised during step 007 of the program. This action, in turn, determines the concentration of the sample in successive wells of the tray 54. For example, to obtain a dilution spectrum in which the concentration in one row is one-half that of the preceding row, the first row of wells might be filled with 100 microliters of the sample and all other wells filled with 50 microliters of diluent each. The microprocessor would be set up to cause 50 microliters to be transferred from one well to the next succeeding well during each cycle.

During step 011, the plunger rods 40 can be oscillated up and down to assure adequate mixing.

At the beginning of each cycle of the serial dilution process, the plunger rods 40 are disposed at a predetermined calibration point within the pipettes. A Hall-effect sensor similar to the type described previously with respect to the table 10 can be used to monitor and control the position of the rods. In step number 014 of the program, after the sample and diluent have been mixed in step 011, the plunger tips are raised so that they are just above the level of liquid in the wells in step 012 and the plunger is returned to the calibration point to expel the liquid from the pipettes in step 013, the tips are raised to a point just above the meniscus of liquid in the receptacle. By then extending the plungers downwardly beyond the calibration point, all liquid is expelled from the pipettes. This action effectively blows the liquid out of the pipettes by causing some air trapped within the pipette to also be ejected and permits any liquid remaining in the tips and extending between the tip and the receptacle to be drawn out of the tip by capillary action due to surface tension acting on the liquid. This step is particularly effective where the tip is made of a non-wettable plastic which as above noted is a preferred material.

Although certain steps have been shown to be discrete, they can be executed simultaneously. For example, steps 016 and 017 might take place at the same time.

Description of Machine Operation

for Tray-to-Tray Transfer

Another important function of the improved liquid handler is tray-to-tray transfer of individual well components. In this procedure, well components from a first tray are picked up by the pipette tips and discharged into the desired wells of a second tray. In the aforementioned series of steps describing serial dilution, transfer of well components to a second tray occurs at step 009 where, after the well components from a row in the first tray are aspirated into the pipette tips (step 007) the table is positioned to bring the desired row of the second tray beneath the pipette tips. The pipettes are then lowered and the contents of the pipettes are expelled into the desired row of wells in the second tray. These steps are repeated as desired to effect the transfer of well components from wells in a first tray to wells in a second tray.

Description of Machine Operation for an

Automated Preparation of Soft Agar Trays

An example of an automated soft agar assay to evaluate the inhibitory effects of various concentrations of a series of three different compounds on the growth of tumorigenic cells is described below. The improved liquid handling machine according to the invention is used to automate the very tedious and otherwise labor intensive process of preparing the many samples to be assayed as required to evaluate the compounds and determine the compound concentration for optimal effectiveness in inhibiting the reproduction of tumorigenic cells.

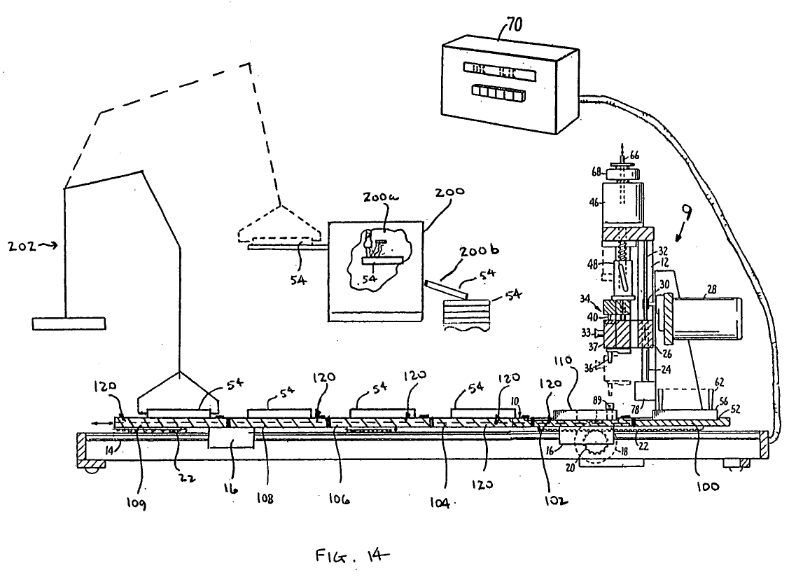

As shown in FIG. 13, the machine 9 for this example has a table 10 with six work stations to prepare the samples for assay. A tip tray 56 holding rows of disposable pipette tips 62 is positioned at the first station, labeled 100. Tip tray 56 is arranged with 12 columns of 8 rows of receptacles 63 (see FIG. 6) holding disposable pipette tips 62. This configuration of 12 columns by 8 rows is chosen to match the culture tray configuration (described below) which will eventually receive the samples to be assayed. There are also 12 pipettes 36 on the head 12 corresponding to the culture tray configuration as well.

A reservoir 110 is positioned at the second station 102. Reservoir 110 has three compartments (see FIG. 12). Compartment 112 initially holds a supply of agarose (a modified agar), compartment 114 initially holds a supply of a growth medium, such as RPMI supplemented with fetal calf serum, and compartment 116 initially holds a mixture of growth medium and tumorigenic cells.

A compound dilution tray 162 is positioned on the third station 104. Tray 162 has 96 wells arranged in 12 columns by 8 rows, the wells being initially empty.

A master compound tray 164 is positioned on the fourth station 106. Tray 164 also has 96 wells arranged in the 12×8 configuration. Three columns of wells contain the initial dilution or highest concentration of the same vehicle, for example, DMSO, in which the compounds are dissolved in the remaining nine columns. These three columns of wells (which do not receive any compound) serve as controls. The remaining nine columns of wells on the master compound tray are divided into three groups of three columns, each group initially holding a different compound at the highest concentration to be evaluated.

A mixing tray 166 is positioned on the fifth station 108. Mixing tray 166 has 96 wells arranged in the 12×8 configuration, the wells being initially empty.

A culture tray 54 is positioned at the sixth station 109. The culture tray 54 has 96 wells arranged in the 12×8 configuration, all of the wells being initially empty.

The process of the preparing trays is controlled by a software program resident in microprocessor 70 and begins with the table 10 being moved to position a row of tips 62 in registration beneath pipettes 36 and lowering the head 12 to load pipette tips on the pipettes. The head is raised and the table is moved to position compartment 114 holding, for example, growth medium beneath the pipettes. The head is lowered dipping the pipettes into the compartment and growth medium is aspirated into the tips. The head is raised and the table is moved to position the pipettes over the wells in the compound dilution tray 162. A predetermined quantity of growth medium is transferred from each pipette into seven of the eight rows of wells in tray 162.

The table is then moved to position the tip tray 56 beneath the pipettes 36 where the used tips are replaced in their wells and a new set of tips is loaded onto the pipettes from the next row in the tray 56. The table is next moved to position the master compound tray 164 beneath the pipettes. The pipettes are lowered into a row of wells in tray 164 whereupon the desired amount of vehicle (first three columns) and compound (remaining nine columns) is aspirated into the tips. The pipettes are raised and the table is moved to position the pipettes above the row of empty wells in the compound dilution tray 162. The pipettes are lowered into the empty wells and compound and vehicle are transferred into them.

The table and head are moved to replace the tips on the pipettes into their receptacles and load a new set of tips onto the pipettes. The table is then moved to position the pipettes over the row of wells in the compound dilution tray 162 which has the vehicle and compounds to be tested. The machine then performs a serial dilution as described above by aspirating a predetermined volume from the first row of wells and transferring the volume into the second row of wells. After a mixing step, the same predetermined volume is aspirated from the second row of wells and transferred to the third row of wells. These steps are repeated for the remaining rows.

Old tips are again exchanged for new tips and the table is moved back and forth to allow the pipettes to aspirate a portion of the compound and vehicle from each well of the compound dilution tray 162 and transfer the portion to a corresponding well of the mixing tray 166.

Pipette tips are again exchanged and the table then positions compartment 116 of the reservoir beneath the pipettes. Compartment 116 holds a mixture of growth medium and tumorigenic cells. The tips are lowered into the compartment and the growth medium and cells are drawn into and expelled from the tips several times to agitate the mixture and ensure that substantially consistent numbers of cells are floating in suspension and will be drawn into the tips. After mixing, cells and growth medium are aspirated into the tips and the table is moved to position the mixing tray 166 beneath the pipettes whereupon the cell and growth medium mixture is transferred into the wells of the mixing tray.

Pipette tips are again exchanged and the table is moved to position the pipettes above compartment 112 of the reservoir which holds the agarose. Agarose is aspirated into the tips and then transferred to the wells of the mixing tray 166 by appropriately positioning table 10.

Tips are again exchanged and then lowered into the wells of the mixing tray, each well having a mixture of agarose, growth medium, cells and vehicle or a compound at a desired concentration. The pipettes are used to mix the contents of the mixing tray by alternately aspirating and expelling the contents from the tips and back into the wells.

In the final step, a portion of the contents of each well in the mixing tray 166 is transferred to a corresponding well in the culture tray 54 at station 109. The mixture in the culture tray is permitted to solidify after which it is incubated at 37° C. in 5% CO₂ atmosphere for a period of time (typically several weeks) while the tumorigenic cells multiply. After the incubation period, the colonies of tumorigenic cells which develop in the culture tray are scored to determine the effect of the compounds at inhibiting the growth of the cells.

As seen by the example of the assay described above, many steps are required to prepare a culture tray having 96 wells to evaluate three inhibitory compounds in triplicate for eight different compound concentrations. Even when the culture trays are prepared by substantially automated methods as described above, the process takes time. If the process is performed at room temperature, and if the temperature of the reservoir 110 and the various trays 162, 164, 166 and 54 are not carefully controlled, then the process will fail because the agarose will solidify in the reservoir and in the wells before it can be transferred and mixed by the action of the pipettes. This problem is exacerbated as the well volume decreases, i.e., the number of wells per tray is increased, as explained above. Therefore, to automate the soft agar assay method it is preferred that the work stations on table 10 have temperature control elements 120 as illustrated in FIG. 12, including the temperature sensors 138, 140, 142, 144, 146 and the associated auxiliary equipment such as power supplies 124, fluid reservoir 128 and heating and cooling devices 130 and 132 or the heat pump 134 as appropriate to the particular heating or cooling element being used. As with all of the other functions of the improved liquid handling machine, the operations of the temperature control elements are controlled by the microprocessor 70 to automatically maintain the temperature of temperature sensitive assay components at the appropriate temperature. For example, in a tumorigenic assay, keeping the agarose at 42° C. and the tumorigenic cells at about 37° C., or in an embryonic stem cell study, keeping stem cells 37° C., or in the study of thermolabile bacteria, keeping the bacteria at their appropriate temperature to simulate the temperature of the hot sulfur vents where the thermolabile bacteria live.

Temperature control is also useful to maintain the cells in the reservoir at a desired optimal temperature to ensure viability of the cells and prevent them from attaching to one another or the walls of the reservoir which cells may do at lower temperatures. Thus, temperature control may ensure sufficient quantities of viable cells for an assay.

Temperature control is also important to maintain the temperature of the agar or agarose, where the ideal temperatures are sufficiently high to prevent premature gelling of the agar or agarose, until the agar or agarose is poured into the well. In the case of Matrigel®, collagen or gelatin, or other similar materials, temperature control is important to prevent premature gelling when the material warms. Therefore, with such materials, it is important to keep the materials chilled prior to filling the wells.

Automated aspects of the assay procedure may be extended to scoring or evaluating the cell colonies in the culture trays to assess the effectiveness of the inhibitory compounds. Several machines may be used in conjunction with the improved liquid handling machine to score the culture trays, as described below.

Fluorescent Scoring

Fluorescent scoring is the currently preferred method of automated evaluation of the cell colonies developed in the soft agar assay. This is because the cells in the samples tend to be dispersed three-dimensionally throughout the agar, and the fluorescent method is sensitive enough to accurately detect colonies which are located deep in the agar.

After the samples in the culture trays have incubated at the appropriate temperature for the appropriate duration and the cells have reproduced to form colonies, a viability compound is inserted into each sample well and allowed to diffuse through the agar. The viability compound, for example, membrane-permeable calcein AM, diffuses into the cells where it is metabolized by the viable cells. During metabolization, the compound is cleaved by natural proteolytic processes and becomes fluorescent. The fluorescent compound is present only in viable cells. The culture trays are placed into the fluorescence scoring machine after the cells have had time to metabolize the viability compound. The machine excites the viability compound in each well of the culture tray with laser light at an appropriate wavelength and the cleaved compound-in the viable cells fluoresces. The fluorescent light emitted by the cleaved compound is detected by a sensor and recorded for each sample in each well. The amount of light emitted from each sample is proportional to the number of viable cells. Generally the control samples which did not see any of the inhibitory compound will have the largest highest of viable cells and, therefore, have the greatest fluorescence. The cell growth in the samples having the inhibitory compounds will generally be less than in the control samples, and the magnitude of the fluorescence from these samples will be some percentage of the fluorescence from the control samples. The fluorescence scoring machine compares the amount of fluorescent light emitted from each well with the amount of light emitted from the wells having the control samples and from these comparisons it is possible to plot data points which represent the percentage of viable cells as a function of compound concentration for each compound tested to determine which concentration yielded the lowest number of viable cells and, therefore, which concentration was most effective at inhibiting the growth of the tumorigenic cells. By having the eight concentrations, in triplicate, inhibition curves may be done as well as statistical analysis.

Spectrophotometric Scoring

Another automated method of evaluating the results of the assay is by spectrophotometric means. Again, a viability compound is inserted into the wells of the culture tray after the incubation period and allowed to diffuse through the agar. The viability compound diffuses into the viable cells where it is cleaved during metabolization. When cleaved, the viability compound assumes a particular color and reflects incident white light at a predominant wavelength. For example, MTT (3-[4,5-dimethylthiazol-2-yl]-2,5-diphenyltetrazolium bromide) viability compound, when cleaved, reflects predominantly red light having a wavelength centered at 570 nm. The machine illuminates each sample in the culture tray with a light source, and a sensor measures the amount of light of the particular wavelength corresponding to the color of the cleaved viability compound which reflects from each well. The amount of reflected light at the wavelength of interest will again be proportional to the number of viable cells in each sample. The machine compares the amount of light reflected at the wavelength of interest from each well with the amount reflected from the wells having the control sample to determine the effectiveness of a particular concentration of a particular inhibitory compound. From this information inhibitory curves can be generated comparing the inhibitory effectiveness as a function of concentration for each compound tested, and the most effective concentration can be ascertained.

Radioactive Scoring

In radioactive scoring, a compound such as thymidine having radioactive tracers such as tritium or radioactive isotopes of sulfur (S35) is placed in each sample well after the incubation period. The viable cells take up the radioactive elements and incorporate them into their DNA and other proteins, thus becoming radioactive. The excess radioactive material not incorporated into the cells is washed out of the wells and the degree of radio activity in each well is then measured by an appropriate sensor such as a Geiger counter. The degree of radio activity is proportional to the viable cells in each well and thus provides a relative measure of the size of the colonies in each well. Again, the control wells whose contents were not subjected to the inhibitory compounds being assayed should have the most viable cells and, therefore, the highest degree of radioactivity as measured by the sensor. Comparison of the degree of radioactivity in the other wells with the control wells provides a relative measure of the effectiveness of the inhibitory compound, and its concentration and this information is used to determine the concentration having maximum effectiveness in inhibiting cancer cell reproduction.

Because the amount of radioactive material taken up by the cells is relatively small, it may be necessary to use a scintillation counter along with a scintillation fluid to detect and amplify the radioactive emissions from the wells to enable the sensors to accurately measure the degree of radioactivity in each well. Scintillation fluids comprise certain organic solutions which fluoresce upon interaction with high energy radiation. The molecules in these solutions are directly or indirectly excited by ionizing radiation, resulting in the emission of photons. These photons are detectable by a photomultiplier device which results in the production of a greatly amplified electron pulse in response to the photons produced by the original ionizing radiation.

The improved liquid handling machine according to the invention can be used in automated scoring of the cell colonies by combining it with any of the above described automated scoring machines.

As shown in FIG. 14, the combined machine incorporates the automated scoring machine 200 with the improved liquid handling machine 9 for performing automated soft agar assays. The operation of the currently preferred embodiment using a fluorescent scoring machine is described below, it being understood that this is by way of example and does not limit the scope of the invention.

In operation of the combined machine, culture trays 54 are removed from the incubator and positioned on the work stations on table 10. The heating and-cooling elements 120 are used to keep the culture trays at a desired temperature to keep the cells in the agarose viable and also to promote cellular metabolic activity so that the viability compound will be readily cleaved once introduced into the cells. Reservoir 10 is filled with a viability compound which fluoresces when cleaved by the cells, and the pipettes 36 are used to transfer a predetermined volume of the viability compound from the reservoir into each well of each culture tray. Once in the wells the viability compound diffuses into the viable cells where it is cleaved. The culture trays 54 remain on the table 10 for a period of time sufficient to permit the viability compound to diffuse and be cleaved, after which the trays are transferred to the fluorescent scoring machine 200. Preferably, this is accomplished by an automated device, such as by a robotic arm 202, which removes each tray 54 from the table 10 (shown in solid line) and inserts them into the scoring machine (shown in dashed line). The colonies in the tray are scored within the machine (illustrated at 200a) by the fluorescent method as described above and the tray is ejected from the scoring machine (illustrated at 200b), which then scores the next tray inserted into the machine.

The improved liquid handling machine according to the invention is expected to realize significant savings in the man-hours required to perform soft agar assays. It is estimated that it would require only 1.5 FTEs (full time employees) to evaluate 15 inhibitory compounds at 8 concentrations in triplicate on five tumorigenic cell lines using the improved method. This evaluation would require preparing 26 trays with the required assay components (for example, filling each well with agarose, growth medium, cells, inhibitory compound or vehicle) and scoring each well after the incubation period. By contrast, using traditional manual methods, this task would take a minimum of 11 FTEs. Thus, the automated procedure afforded by the improved liquid handling machine and method according to the invention can be expected to reduce the time required to perform soft agar assays by a factor of 10 which should lead to significant cost savings, decrease the time required to evaluate compounds of interest, and increase the consistency in scoring by eliminating the human variability factor. The present invention also permits miniaturization of soft agar assays, as well as other assays that use temperature sensitive components.

What is claimed is:

1. An improved liquid handling machine for automatically transferring liquid between a plurality of liquid containing wells, said machine having:
a head;
a plunger assembly mounted on said head, said plunger assembly including a plurality of pipettes having depending ends for receiving tips, a plurality of plungers respectively disposed within said pipettes, said plungers being movable coaxially within said pipettes to vary their internal volumes;
a tip ejector for removing tips disposed on said depending ends of said pipettes;
a support mounted beneath said head, said support having at least a first work station being adapted to accommodate a tray having pipette tip-receiving receptacles, a second and a third work station each being adapted to accommodate a respective tray of said liquid containing wells;
said support and said head being movable relatively to one another to place said pipettes in registry with any of said tips in said tip-receiving receptacles and any of said wells in registry with said pipettes;
a motion controller for controlling the relative motion between said head and said support as well as the motion of said plungers and said tip ejector to effect transfer of liquid between the liquid containing wells and to replace tips on the ends of said pipettes with other tips disposed in at least some of the pipette tip-receiving receptacles between predetermined liquid transfer steps, wherein the improvement comprises:
said support having a plurality of temperature control elements for independently controlling the temperature at each of said second and third work stations to maintain said liquid in said liquid containing wells at a predetermined temperature, said support also having a sensor at each of said second and third work stations for sensing the temperature, and a temperature controller for controlling said temperature control elements and said sensors.

2. An improved liquid handling machine according to claim 1, wherein said support comprises a table having a surface for supporting said trays.

3. An improved liquid handling machine according to claim 2, wherein said head is movably mounted for translational motion along an axis substantially perpendicular to the surface of said table.

4. An improved liquid handling machine according to claim 3, wherein said table is movably mounted for translational motion along a path in a horizontal plane, said table being elongated in a direction parallel to said path, said work stations being spaced on said table along said path.

5. An improved liquid handling machine according to claim 4, further comprising an insulating strip for thermally isolating said second and third work stations from one another, said insulating strip being interposed between said second and third work stations.

6. An improved liquid handling machine according to claim 4, wherein said temperature control elements comprise independent heaters for heating said table at each of said second and third work stations.

7. An improved liquid handling machine according to claim 4, wherein said temperature control elements comprise independent cooling devices for cooling said table at each of said second and third work stations.

8. An improved liquid handling machine according to claim 6, wherein one of said heaters comprises an electrical resistive heating element positioned at one of said second and third work stations.

9. An improved liquid handling machine according to claim 6, wherein one of said heaters comprises:
a passageway arranged in said table beneath one of said second and third work stations;
a fluid movable through said passageway;
a pump connected with said passageway for moving said fluid therethrough;
a heating device for heating said fluid; and
said fluid being heated as required to maintain the temperature of said liquid sample in said wells at said one work station at said predetermined temperature, heat being transferred between said fluid and said liquid sample by conduction through said table and said tray containing said liquid.

10. An improved liquid handling machine according to claim 9, wherein said heating device comprises a heat pump.

11. An improved liquid handling machine according to claim 6, wherein said heater comprises a Peltier effect module.

12. An improved liquid handling machine according to claim 7, wherein one of said cooling devices comprises:
a passageway arranged in said table beneath one of said second and third work stations;
a fluid movable through said passageway;
a pump connected with said passageway for moving said fluid therethrough;
a cooler module for cooling said fluid; and
said fluid being cooled as required to maintain the temperature of said liquid sample in said wells at said one work station at said predetermined temperature, heat being transferred between said fluid and said liquid sample by conduction through said table and said tray containing said liquid.

13. An improved liquid handling machine according to claim 12, wherein said cooler module comprises a heat pump.

14. An improved liquid handling machine according to claim 7, wherein said cooling device comprises a Peltier effect module.

15. An improved liquid handling machine according to claim 1, wherein said temperature controller comprises a microprocessor.

16. An improved liquid handling machine according to claim 1, wherein said motion controller comprises a microprocessor.

17. An improved liquid handling machine according to claim 1, further comprising a scoring device for evaluating results of an assay of said liquid in said wells, and a transporter for transferring said tray of said liquid containing wells from one of said second and third work stations to said scoring device.

18. An improved liquid handling machine according to claim 17, wherein said scoring device comprises a fluorescence scoring machine.

19. An improved liquid handling machine according to claim 17, wherein said scoring device comprises a spectrophotometric scoring machine.

20. An improved liquid handling machine according to claim 17, wherein said transporter comprises a robotic arm.

21. An assay method determining the effect of a test compound on a cell in a predetermined well comprising semi-solid matrix, comprising:
(a) using a liquid handler with a first reservoir and a second reservoir to transfer a liquid semi-solid matrix from the first reservoir to a predetermined well of an assay plate;
(b) using a liquid handler to transfer cells from a second reservoir to the predetermined well of an assay plate;
(c) allowing the liquid semi-solid matrix in the predetermined well to solidify;
(d) incubating the cells in the plate for a period of time for the cells to grow into a colony;
(e) adding a predetermined amount of a test compound to the predetermined well; and
(f) determining the biological effect of the test compound on the colony.

22. The assay method of claim 21, wherein the semi-solid matrix solidifies between 10 to 45 degrees Celsius.

23. The assay method of claim 22, wherein the semi-solid matrix solidifies between 15 to 40 degrees Celsius.

24. The assay method of claim 23, wherein the semi-solid matrix solidifies between 20 to 35 degrees Celsius.

25. The assay method of claim 24, wherein the semi-solid matrix solidifies between 20 to 30 degrees Celsius.

26. The assay method of claim 21, wherein the semi-solid matrix comprises agar, agarose, collagen, or basement membrane.

27. The assay method of claim 26, wherein the semi-solid matrix comprises agar, agarose, or collagen.

28. The assay method of claim 27, wherein the semi-solid matrix comprises agar or agarose.

29. The assay method of claim 28, wherein the semi-solid matrix comprises agarose.

30. The assay method of claim 21, wherein the semi-solid matrix is heated until it is a liquid before the liquid semi-solid matrix is added to the first reservoir.

31. The assay method of claim 21, wherein the semi-solid matrix is heated until it is a liquid after the semi-solid matrix is added to the first reservoir.

32. The assay method of claim 21, wherein the semi-solid matrix comprises collagen or Matrigel®.

33. The assay method of claim 32, wherein the semi-solid matrix comprises Matrigel®.

34. The assay method of claim 21, wherein the semi-solid matrix is cooled until it is a liquid before the liquid semi-solid matrix is added to the first reservoir.

35. The assay method of claim 21, wherein the semi-solid matrix is cooled until it is a liquid after the semi-solid matrix is added to the first reservoir.

36. An assay method for determining the effect of a test compound on cells comprising:
(a) using a liquid handler to transfer a liquid semi-solid matrix from a first reservoir to a predetermined well of an assay plate;
(b) using a liquid handler to transfer growth medium from a second reservior to the predetermined well of the assay plate;
(c) using a liquid handler to transfer cells from a third reservoir to the predetermined well of the assay plate;
(d) allowing the liquid semi-solid matrix in the predetermined well of the assay plate to solidify;
(e) incubating the cells in the predetermined well of the assay plate for a period of time for the cells to grow into a colony;
(f) adding a predetermined amount of a test compound to the predetermined well of the assay plate; and
(g) determining the biological effect of the test compound on the colony.

37. An assay method for determining the effect of a test compound on cells comprising:
(a) using a liquid handler to transfer a liquid semi-solid matrix from a first reservoir into a predetermined well of the assay plate;
(b) using a liquid handler to transfer growth medium from a second reservior to the predetermined well of the assay plate;
(c) using a liquid handler to transfer cells from a third reservoir to the predetermined well of the assay plate;
(d) using a liquid handler to transfer a predetermined amount of a test compound from a fourth reservoir to the predetermined well of the assay plate;
(e) allowing the liquid semi-solid matrix in the predetermined well of the assay plate to solidify;
(f) incubating the cells in the predetermined well of the assay plate for a period of time for the cells to grow to form a colony; and
(g) determining the biological effect of the test compound on the colony.

38. The assay method of claim 37, wherein the liquid handler is used to prepare dilutions of the test compound in predetermined wells in a dilution plate.

39. The assay method of claim 38, wherein the liquid handler transfers a predetermined amount of test compound from a predetermine well in the dilution plate to the predetermined well of the assay plate.

40. The assay method of claim 21, wherein the assay plate comprises six or more wells.

41. The assay method of claim 40, wherein the assay plate comprises twenty-four or more wells.

42. The assay method of claim 41, wherein the assay plate comprises forty-eight or more wells.

43. The assay method of claim 42, wherein the assay plate comprises ninty-six or more wells.

44. The assay method of claim 43, wherein the assay plate comprises 384 or more wells.

45. The assay method of claim 21, wherein the cells are capable of non-adherent growth in a semi-solid matrix.

46. The assay method of claim 45, wherein the cells are normal primary cells, stem cells, or tumor cells.

47. The assay method of claim 46, wherein the cells are normal primary cells.

48. The assay method of claim 46, wherein the cells are stem cells.

49. The assay method of claim 46, wherein the cells are tumor cells.

50. The assay method of claim 49, wherein the tumor cells are breast tumor cells, ovarian tumor cells, melanoma cells, neuroblastoma cells, colon tumor cells, prostate tumor cells, large cell lung tumor cells or small cell lung tumor cells.

51. The assay method of claim 21, wherein the effect of the test compound on the colony is determined by a use of a luminometer, use of a photometer, use of scintillation, use of fluorescence, or by visual counting of colonies.

52. The assay method of claim 28, wherein the effect of the test compound on the colony is determined by use of fluorescence or by use of a luminometer.

53. The assay method of claim 21, wherein the effect of the test compound on the colony is determined by a viability stain.

54. The assay method of claim 53, wherein the viability stain is a fluorescent probe.

55. The assay method of claim 53, wherein the viability stain is Calcein AM.

56. The assay method of claim 21, wherein the liquid handler is capable of transfering a volume of a liquid from about a nanoliter to about five microliters to a predetermined well.

57. The assay method of claim 56, wherein the liquid handler is capable of transfering a volume of a liquid from about one microliter to about five hundred microliter to a predetermined well.

58. The assay method of claim 21, wherein the liquid handler is capable of maintaining a predetermined temperature of a reservoir.

59. The assay method of claim 21, wherein the assay plate is a microtube.

60. The assay method of claim 21, wherein the liquid handler is capable of simultaneous transfer of a liquid to more than one well.

61. The assay method of claim 21, wherein the semi-solid matrix comprises at least 25 percent agar, agarose, collagen, or basement membrane.

62. The assay method of claim 61, wherein the semi-solid matrix comprises at least 45 percent agar, agarose, collagen, or basement membrane.

63. The assay method of claim 62, wherein the semi-solid matrix comprises at least 60 percent agar, agarose, collagen, or basement membrane.

64. The assay method of claim 63, wherein the semi-solid matrix comprises at least 75 percent agar, agarose, collagen, or basement membrane.

65. The assay method of claim 21, wherein the semi-solid matrix comprises at least 25 percent agarose.

66. The assay method of claim 65, wherein the semi-solid matrix comprises at least 45 percent agarose.

67. The assay method of claim 66, wherein the semi-solid matrix comprises at least 60 percent agarose.

68. The assay method of claim 69, wherein the semi-solid matrix comprises at least 75 percent agarose.

69. The assay method of claim 21, wherein the semi-solid matrix comprises at least 25 percent agar.

70. The assay method of claim 69, wherein the semi-solid matrix comprises at least 45 percent agar.

71. The assay method of claim 70, wherein the semi-solid matrix comprises at least 60 percent agar.

72. The assay method of claim 71, wherein the semi-solid matrix comprises at least 75 percent agar.

73. The assay method of claim 61, wherein the assay plate comprises from 50 wells to 2000 wells.

74. The assay method of claim 73, wherein the assay plate comprises from 50 wells to 200 wells.

75. The assay method of claim 74, wherein the assay plate comprises from 200 wells to 500 wells.

76. The assay method of claim 75, wherein the assay plate comprises from 300 wells to 500 wells.

77. The assay method of claim 73, wherein the assay plate comprises from 350 wells to 2000 wells.

78. The assay method of claim 21, wherein the liquid handler is an automated system.

79. The assay method of claim 21, wherein the effect of the test compound on the colony is determined by the cell viability.

80. The assay method of claim 79, wherein the cell viability to determined by a fluorescent probe.

81. A method of using a liquid handler for automated preparation of an assay plate comprising wells with a semi-solid matrix, wherein
the assay plate comprises multiple wells in one or more columns, wherein at least one well is capable of holding a semi-solid matrix,
the liquid handler comprises a first reservoir comprising growth medium at one concentration, second reservoir comprising a liquid semi-solid matrix, third reservoir comprising cells in a liquid, and fourth reservoir, and the means to transfer a liquid from a reservoir to a predetermined well in an assay plate, by
a) transferring a predetermined amount of growth medium from the first reservoir to the fourth reservoir,
b) transferring a predetermined amount of the liquid semi-solid matrix from the second reservoir to the fourth reservoir,
c) transferring a predetermined amount of cells in a liquid from the third reservoir to the fourth reservoir,
d) mixing the growth medium, liquid semi-solid matrix and cells in the fourth reservoir,
e) transferring a predetermined amount of the mixed growth medium, liquid semi-solid matrix and cells from the fourth reservoir into a predetermined well of the first assay plate, and
f) allowing the semi-solid matrix to solidify,
wherein steps a-c may be performed in any order.

82. The method of claim 81, wherein the first reservoir, second reservoir, third reservoir or fourth reservoir comprises a test compound.

83. The method of claim 82, wherein the first reservoir, second reservoir, or third reservoir comprises a test compound.

84. The method of claim 83, wherein the first reservoir comprises a test compound.

85. The method of claim 83, wherein the second reservoir comprises a test compound.

86. The method of claim 83, wherein the third reservoir comprises a test compound.

87. The method of claim 83, wherein the fourth reservoir comprises a test compound.

88. The method of claim 81, comprising the additional step g) incubating the assay plate for a period of time under conditions for the cells to form a colony.

89. The method of claim 88, comprising the additional step h) determining the effect of the test compound on the colony.

90. A method of using a liquid handler for automated preparation of an assay plate comprising wells with a semi-solid matrix, wherein
the assay plate comprises multiple wells in one or more columns, wherein at least one well is capable of holding a semi-solid matrix,
the liquid handler comprises a first reservoir comprising growth medium, a liquid semi-solid matrix, and cells, and second reservoir, and the means to transfer a liquid from a reservoir to a predetermined well in an assay plate, by
a) mixing the growth medium, liquid semi-solid matrix and cells in the first reservoir,
b) transferring a predetermined amount of the mixed growth medium, liquid semi-solid matrix and cells from the first reservoir into a predetermined well of the first assay plate, and
c) allowing the semi-solid matrix in the predetermined well to solidify,
optionally repeating steps a and b, but transferring into a second predetermined well of the first assay plate.

91. The method of claim 90, comprising the additional step g) incubating the assay plate for a period of time under conditions for the cells to form a colony.

92. The method of claim 91, comprising the additional step h) adding a test compound to the predetermined well

93. The method of claim 92, comprising the additional step i) determining the effect of the test compound on the colony.

94. A method of using a liquid handler for automated preparation of an assay plate comprising wells with a semi-solid matrix, wherein
the assay plate comprises multiple wells in one or more columns, wherein at least one well is capable of holding a semi-solid matrix,
the liquid handler comprises a first reservoir compriseing growth medium at one concentration, second reservoir comprising a liquid semi-solid matrix, third reservoir comprising cells in a liquid, and fourth reservoir, and the means to transfer a liquid from a reservoir to a predetermined well in an assay plate,
1. by forming a first layer of a semi-solid matrix in a predetermined well of a first assay plate, comprising,
a) transferring a predetermined amount of the liquid semi-solid matrix from the second reservoir to a predetermined well in the assay plate,
b) allowing the semi-solid matrix to solidify,
2. by forming a second layer of a semi-solid matrix, growth medium and cells in a predetermined well of a first assay plate, comprising,
a) transferring a predetermined amount of growth medium from the first reservoir to the fourth reservoir,
b) transferring a predetermined amount of the liquid semi-solid matrix from the second reservoir to the fourth reservoir,
c) transferring a predetermined amount of cells in a liquid from the third reservoir to the fourth reservoir,
d) mixing the growth medium, liquid semi-solid matrix and cells in the fourth reservoir,
c) transferring a predetermined amount of the mixed growth medium, liquid semi-solid matrix and cells from the fourth reservoir into a predetermined well of the first assay plate, and
f) allowing the second layer of liquid semi-solid matrix to solidify, optionally repeating steps 1 and/or 2, but transferring into a second predetermined well of the first assay plate.

95. A method for using a liquid handler for automated preparation of an assay plate containing semi-solid matrix, wherein
each plate has at least one well in one or more columns capable of holding a semi-solid matrix,
the liquid handler comprises a first reservoir comprising a liquid growth medium in one concentration, a second reservoir a liquid growth medium in second concentration, a third reservoir comprising a liquid growth medium in third concentration, a fourth reservoir containing liquid semi-solid matrix, and a fifth reservoir comprising cells in liquid growth medium at the first concentration; by
1) preparing a first layer of semi-solid matrix in a predetermined well in a first assay plate, comprising
a) transferring a predetermined amount of growth medium from the third reservoir to the liquid semi-solid matrix in a fourth reservoir, to form a liquid mixture,
b) transferring a predetermined amount of liquid mixture from the fourth reservoir to a predetermined well in first assay plate,
c) allowing the liquid mixture in the predetermined well in the first assay plate to solidify,
d) optionally repeating steps b and c with a second predetermined well in the first assay plate;
2) preparing a second layer of semi-solid matrix in a predetermined well in the first assay plate, comprising
a) transferring a predetermined amount of the growth medium from the second reservoir to the fifth reservoir comprsing the cells,
b) transferring a predetermined amount of liquid semi-solid matrix in the fourth reservoir to the fifth reservoir,
c) mixing contents of the fifth reservoir,
d) transferring a predetermined amount of the mixure in the fifth reservoir to first layer of semi-solid matrix in a predetermined well in a first assay plate to form a second layer,
e) allowing the second layer in the predetermined well to solidify.

96. The method of claim 95, comprising the additional step g) incubating the assay plate for a period of time under conditions for the cells to form a colony.

97. The method of claim 96, comprising the additional step h) adding a test compound to the predetermined well

98. The method of claim 97, comprising the additional step i) determining the effect of the test compound on the colony.

99. The method of claim 95, comprising the further step of preparing a dilution plate comprising a first predetermined well comprising a test compound, by
a) transferring a predetermined amount of the growth medium from a first reservoir to a second predetermined well in the dilution plate,
b) transferring a predetermined amount of test compound from the first predetermined well into second predetermined well in the dilution plate,
c) mixing the contents of the second dilution well,
d) repeating steps a, b and c, but to a third predetermined well in the dilution plate.
